(19) United States
(12) Patent Application Publication
(10) Pub. No.: US 2008/0028487 A1
(43) Pub. Date: Jan. 31, 2008

(54) USES OF THE PSEUDOMONAS SYRINGAE EFFECTOR PROTEIN HopU1 RELATED TO ITS ABILITY TO ADP-RIBOSYLATE EUKARYOTIC RNA BINDING PROTEINS

(75) Inventors: JAMES R. ALFANO, Lincoln, NE (US); ZHENG QING FU, Lincoln, NE (US); THOMAS E. ELTHON, Lincoln, NE (US)

Correspondence Address: MCKEE, VOORHEES & SEASE, P.L.C., 801 GRAND AVENUE, SUITE 3200, DES MOINES, IA, 50309-2721, US

(73) Assignee: THE UNIVERSITY OF NEBRASKA-LINCOLN, LINCOLN, NE (US)

(21) Appl. No.: 11/759,054

(22) Filed: Jun. 6, 2007

Related U.S. Application Data
(60) Provisional application No. 60/804,068, filed on Jun. 6, 2006.

(51) Int. Cl. A01H 1/00; C12Q 1/68

(52) U.S. Cl. 800/278; 435/6

(57) ABSTRACT

The bacterial plant pathogen Pseudomonas syringae injects effector proteins into host cells via a type III protein secretion system to cause disease. The invention relates to the discovery that the type III effector HopU1 is a mono-ADP-ribosyltransferase (ADP-RT) and suppresses plant innate immunity. The HopU1 substrates in Arabidopsis thaliana extracts were RNA-binding proteins that possess RNA-recognition motifs (RRMs). A. thaliana knock-out lines defective in the glycine-rich RNA-binding protein AtGRP7, a HopU1 substrate, were more susceptible than wild type plants to P. syringae. The ADP-ribosylation of AtGRP7 by HopU1 required two arginines within the RRM. The invention provides novel methods for the modulation of the innate immune response of a plant to a biotic stress, including methods for enhancing or suppressing the innate immune response of the plant.

### CROSS-REFERENCE TO RELATED APPLICATIONS

This application claims priority under 35 U.S.C. § 119 of a provisional application Ser. No. 60/804,068 filed Jun. 6, 2006, which application is hereby incorporated by reference in its entirety.

### GRANT REFERENCE

This invention was made with government support under NSF Grant No. DBI-0077622, NSF Grant No. MCB-0317165, and NIH Grant No. 1R56AI069146-01. The Government has certain rights in the invention.

### FIELD OF THE INVENTION

The present invention provides methods for the production of plants with enhanced or suppressed innate immune responses to a biotic stress.

### BACKGROUND OF THE INVENTION

Interactions between bacterial pathogens and their plant hosts generally fall into two categories: (1) compatible (pathogen-host), leading to intercellular bacterial growth, symptom development, and disease development in the host plant; and (2) incompatible (pathogen-nonhost), resulting in the hypersensitive response, a particular type of incompatible interaction occurring, without progressive disease symptoms. During compatible interactions on host plants, bacterial populations increase dramatically and progressive symptoms occur. During incompatible interactions, bacterial populations do not increase, and progressive symptoms do not occur.

The hypersensitive response (“HR”) is a rapid, localized necrosis that is associated with the active defense of plants against many pathogens (Horsfall et al., eds., Plant Disease: An Advanced Treatise Vol. 5, pp. 201 224, New York, N.Y.: Academic Press (1980); Mount et al., eds., Phytopathogenic Prokaryotes, Vol. 2, pp. 149 177, New York, N.Y.: Academic Press (1982)). The hypersensitive response elicited by bacteria is readily observed as a tissue collapse if high concentrations (˜10⁷ cells/ml) of a limited host-range pathogen like Pseudomonas syringae or Erwinia amylovora are infiltrated into the leaves of nonhost plants (necrosis occurs only in isolated plant cells at lower levels of inoculum) (Klement, Nature 199:299-300 (1963); Klement et al., Phytopathology 54:474-477 (1963); Turner et al., Phytopathology 64:885-890 (1974); Mount et al., eds., Phytopathogenic Prokaryotes, Vol. 2., pp. 149-177, New York, N.Y.: Academic Press (1982)). The capacities to elicit the hypersensitive response in a nonhost and be pathogenic in a host appear linked. As noted by Mount et al., eds., Phytopathogenic Prokaryotes, Vol. 2., pp. 149 177, New York, N.Y.: Academic Press (1982), these pathogens also cause physiologically similar, albeit delayed, necroses in their interactions with compatible hosts. Furthermore, the ability to produce the hypersensitive response or pathogenesis is dependent on a common set of genes, denoted hrp (Lindgren et al., J. Bacteriol. 168:512-22 (1986); Willis et al., Mol. Plant-Microbe Interact. 4:132-138 (1991)). Consequently, the hypersensitive response may hold clues to both the nature of plant defense and the basis for bacterial pathogenicity.

The hrp genes are widespread in gram-negative plant pathogens, where they are clustered, conserved, and in some cases interchangeable (Willis et al., Mol. Plant-Microbe Interact. 4:132-138 (1991); Dangl, ed., Current Topics in Microbiology and Immunology: Bacterial Pathogenesis of Plants and -Molecular and Cellular Mechanisms, pp. 79-98, Berlin: Springer-Verlag (1994)). Several hrp genes encode components of a protein secretion pathway similar to one used by Yersinia, Shigella, and Salmonella spp. to secrete proteins essential in animal diseases (Van Gijsegem et al., Trends Microbiol. 1:175-180 (1993)). In E. amylovora, P. syringae, and P. solanacearum, hrp genes have been shown to control the production and secretion protein elicitors of the hypersensitive response (He et al., Cell 73:1255-1266 (1993); Wei et al., J. Bacteriol. 175:7958-7967 (1993); Arlat et al., EMBO J. 13:543-553 (1994)). Hypersensitive response elicitor proteins, designated harpins, are proteins found in phytopathogens containing a type III secretion system and are typically glycine-rich, acidic, cysteine-lacking, heat stable proteins (He et al., Cell 73: 1255-1266 (1993).

The first of these proteins was discovered in E. amylovora Ea321, a bacterium that causes fire blight of rosaceous plants, and was designated harpin (Wei et al., Science 257:85-88 (1992)). Mutations in the encoding hrpN gene revealed that harpin is required for E. amylovora to elicit a hypersensitive response in nonhost tobacco leaves and incite disease symptoms in highly susceptible pear fruit. The P. solanacearum GMI1000 PopA1 protein has similar physical properties and also elicits the hypersensitive response in leaves of tobacco, which is not a host of that strain (Arlat et al., EMBO J. 13:543-53 (1994)). However, P. solanacearum popA mutants still elicit the hypersensitive response in tobacco and incite disease in tomato. Thus, the role of these glycine-rich hypersensitive response elicitors can vary widely among gram-negative plant pathogens.

Other plant pathogenic hypersensitive response elicitors have been isolated, cloned, and sequenced from various organisms, including: HrpW from Erwinia amylovora (Kim et al., J. Bacteriol. 180(19):5203-5210 (1998)); HrpN from Erwinia chrysanthemi (Bauer et al., MPMI 8(4):484-91 (1995)); HrpN from Erwinia carotovora (Cui et al., MPMI 9(7):565-73 (1996)); HrpN from Erwinia stewartii (Ahmad et al., 8th Int'l. Cong. Molec. Plant-Microb. Inter. Jul. 14-19, 1996 and Ahmad et al., Ann. Mtg. Am. Phytopath. Soc. Jul. 27-31, 1996); hreX from Xanthomonas campestris (U.S. Patent Application Publ. No. 20020066122 to Wei et al.); HrpZ from Pseudomonas syringae pv. syringae (He et al., Cell 73:1255-1266 (1993); WO 94/26782 to Cornell Research Foundation, Inc.); and HrpW from Pseudomonas syringae pv. tomato (Charkowski et al., J. Bacteriol. 180:5211-5217 (1998)).

Many gram-negative pathogens of plants and other eukaryotic-associated bacteria utilize type III protein secretion systems (T3SSs). (He, S. Y., Nomura, K. & Whittam, T. S. Type III protein secretion mechanism in mammalian and plant pathogens. Biochim. Biophys. Acta 1694:181-206 (2004); and Galan, J. E. & Wolf-Watz, H. Protein delivery into eukaryotic cells by type III secretion machines. Nature 444:567-573 (2006)). T3SSs are molecular syringes that inject bacterial proteins called effectors into eukaryotic host cells to modulate host physiology. In animal cells their activities alter specific host cell functions including phagocytosis, proinflammatory responses, apoptosis, and intracellular trafficking (Mota, L. J. & Cornelis, G. R. The bacterial injection kit: type III secretion systems. Ann. Med. 37:234-249 (2005)). Much less is understood about the activities and targets of type III effectors from plant pathogens. The emerging picture is that many type III effectors from plant pathogens suppress host immune responses (Abramovitch, R. B., Anderson, J. C. & Martin, G. B. Bacterial elicitation and evasion of plant innate immunity. Nat. Rev. Mol. Cell. Biol. 7:601-611 (2006); Espinosa, A. & Alfano, J. R. Disabling surveillance: bacterial type III secretion system effectors that suppress innate immunity. Cell. Microbiol. 6:1027-1040 (2004); and Mudgett, M. B. New insights to the function of phytopathogenic bacterial type III effectors in plants. Annu. Rev. Plant Biol. 56:509-531 (2005)). Thus far, effectors that possess cysteine protease, tyrosine phosphatase and E3 ubiquitin ligase activities have been implicated in plant innate immunity suppression (Axtell, M. J. & Staskawicz, B. J. Initiation of RPS2-specified disease resistance in Arabidopsis is coupled to the AvrRpt2-directed elimination of RIN4. Cell 112:369-377 (2003); Mackey, D., Belkhadir, Y., Alonso, J. M., Ecker, J. R. & Dangl, J. L. Arabidopsis RIN4 is a target of the type III virulence effector AvrRpt2 and modulates RPS2-mediated resistance. Cell 112:379-389 (2003); Shao, F. et al. Cleavage of Arabidopsis PBS1 by a bacterial type III effector. Science 301:1230-1233 (2003); Lopez-Solanilla, E., Bronstein, P. A., Schneider, A. R. & Collmer, A. HopPtoN is a Pseudomonas syringae Hrp (type III secretion system) cysteine protease effector that suppresses pathogen-induced necrosis associated with both compatible and incompatible plant interactions. Mol. Microbiol. 54:353-365 (2004); Bretz, J. R. et al. A translocated protein tyrosine phosphatase of Pseudomonas syringae pv. tomato DC3000 modulates plant defence response to infection. Mol. Microbiol. 49:389-400 (2003); Espinosa, A., Guo, M., Tam, V. C., Fu, Z. Q. & Alfano, J. R. The Pseudomonas syringae type III-secreted protein HopPtoD2 possesses protein tyrosine phosphatase activity and suppresses programmed cell death in plants. Mol. Microbiol. 49:377-387 (2003); and Janjusevic, R., Abramovitch, R. B., Martin, G. B. & Stebbins, C. E. A bacterial inhibitor of host programmed cell death defenses is an E3 ubiquitin ligase. Science 311:222-226 (2006)), however, the enzymatic activities for most plant pathogen type III effectors that suppress innate immunity remain unknown.

### BRIEF SUMMARY OF THE INVENTION

The present invention relates to a method of identifying a substrate of HopU1. In one aspect, the identification is performed in vitro or in vivo. In one aspect, the substrate is identified from or in a plant or animal cell or extract. Also included are methods for enhancing the innate immune response of a plant. In one aspect, the method includes over-expressing a HopU1 substrate in a plant cell. In another aspect, the method includes methods for suppressing the innate immune response of a plant. In one aspect, the method includes expressing HopU1 in a plant cell.

### BRIEF DESCRIPTION OF THE DRAWINGS

FIG. 1. HopU1 is a putative mono-ADP-ribosyltransferase that contributes to virulence. (a) Alignment of the conserved regions of known mono-ADP-ribosyltransferases (ADP-RTs) with putative DC3000 ADP-RTs. Conserved residues are shown underlined and the invariant amino acids of the CT group of ADP-RTs (Sun, J. & Barbieri, J. T. Pseudomonas aeruginosa ExoT ADP-ribosylates CT10 regulator of kinase (Crk) proteins. J. Biol. Chem. 278, 32794-32800 (2003)) are marked with asterisks. (b) hopU1 (white box) is downstream of a type III-related promoter, the shcF type III chaperone gene, and the hopF2 effector gene (hatched boxes). (c) RNA was isolated from DC3000 (WT) or the ΔhopU1 mutant grown in either rich media (1) or a minimal medium that induces type III-related genes (2) and used in RT-PCR reactions. A DNA control (C) and no RT controls (No RT) were included.

FIG. 2. HopU1 suppresses outputs of plant innate immunity. (a) DC3000, ΔhopU1 mutant, and the ΔhopU1 mutant expressing hopU1 were infiltrated into tobacco leaves at threshold cell densities (1×10⁶ cells/ml). After 24 h the tissue was assessed for HR production. (b) The ΔhopU1 mutant carrying a vector control (pvector), a hopU1 construct (phopU1), or a hopU1 ADP-RT catalytic mutant construct (phopU1_{DD}) were infiltrated into tobacco at 1×10⁷ cells/ml and assessed for HR production after 24 h. (c) Callose deposition was visualized in A. thaliana plants expressing HopU1-HA or the HopU1_{DD}-HA mutant 16 h after treatment with flg22. (d) Callose deposition was quantified by counting the number of callose foci per field of view. Twenty leaf regions (4 fields of view from 5 different leaves) were averaged and error bars (s.e.m.) are indicated. The experiment was repeated three times.

FIG. 3. HopU1-His ADP-ribosylates poly-L-arginine and proteins in Arabidopsis and tobacco. (a) SDS-PAGE of partially purified HopU1-His and HopU1-His catalytic mutant (HopU1_{DD}-His) used in ADP-RT assays. (b) HopU1-His (stippled bar), HopU1_{DD}-His (white bar), and a BSA control (black bar) were incubated with poly-L-arginine in the presence of [³²P]-NAD. ³²P-labeled products were quantified with liquid scintillation. The specific activity unit is μmole ³²P transferred/min/μmole of HopU1 times 10⁻¹⁰. The experiment was performed twice and the standard errors are indicated. (c) Autoradiograms of ADP-RT assays with either Arabidopsis (At) or tobacco (Nt) extracts with HopU1-His or HopU1_{DD}-His.

FIG. 4. HopU1-His ADP-ribosylates recombinant RNA-binding proteins in vitro and in planta. (a) Recombinant CP-RBP and GR-RBP glutathione S-transferase (GST) fusions and a GST control were used in ADP-RT assays with HopU1-His. ADP-RT reactions were subjected to SDS-PAGE followed by autoradiography. The substrate fusions are listed with their A. thaliana locus or protein name. (b) Schematic representation of the RRM and glycine-rich domains within AtGRP7 and the locations of the arginine residues (R) throughout the protein. Black and grey sections within the RRM domain depict RNP1 and RNP2, respectively, two conserved regions within RRM domain (Burd, C. G. & Dreyfuss, G. Conserved structures and diversity of functions of RNA-binding proteins. Science 265, 615-621 (1994)). The locations of the arginine residues (R) are indicated. (c) In vitro ADP-RT assays were done with HopU1-His and various AtGRP7-GST fusions. These included an RRM-GST fusion, a glycine-rich domain-GST fusion, and AtGRP7-GST mutants that have each arginine within the RRM domain separately substituted with lysine. Autoradiograms (top panel) indicate the AtGRP7-GST derivatives that maintained the ability to be ADP-ribosylated and immunoblots (bottom panel) indicate the relative stability of each AtGRP7-GST derivative. (d) AtGRP7 fused to an HA epitope (AtGRP7-HA) or an AtGRP7-HA derivative that is unable to be ADP-ribosylated (AtGRP7R_{47K}-HA) were transiently expressed in N. benthamiana with HopU1-FLAG or the ADP-RT catalytic mutant HopU1_{DD}-FLAG. After 40 h leaf samples were subjected immunoblot analysis using anti-HA and anti-FLAG antibodies. (a,c) Molecular mass markers (kDa) are indicated.

FIG. 5. Analyses of A. thaliana Atgrp7-1 mutant plants suggest AtGRP7 plays a role in innate immunity. (a) Disease symptoms in Atgrp7-1 plants and A. thaliana Col-0 plants after spray-inoculation with at a cell density of 2×10⁸ cells/ml. Pictures were taken after 5 days. The experiments in ‘b’ and ‘c’ were repeated five times with similar results. (b) Bacterial growth assays of wild type DC3000 and the ΔhrcC mutant spray-inoculated at a cell density of 2×10⁸ cells/ml onto Atgrp7-1 and wild type A. thaliana Col-0 plants. (c) Callose deposition was determined in A. thaliana Col-0 and Atgrp7-1 mutant plants 16 h after treatment with flg22. The number of callose foci per field of view for 20 leaf regions (4 fields of view from 5 different leaves) were averaged ±s.e.m. The experiment was repeated three times with similar results.

FIG. 6. HopU1 is injected by the DC3000 T3SS and contributes to virulence. (a) Adenylate cyclase (CyaA) assays were carried out by infiltrating DC3000 and a ΔhrcC mutant (defective in T3SS) carrying constructs that produced HopU1-CyaA, HopU1_{DD}-CyaA (HopU1 catalytic mutant), or AvrPto1-Cya (a type III effector known to be injected), respectively, into Nicotiana benthamiana. cAMP levels were determined 10 h after infiltration. (b) Bacterial strains were inoculated into A. thaliana Col-0 leaves by dipping plants into bacterial suspensions.

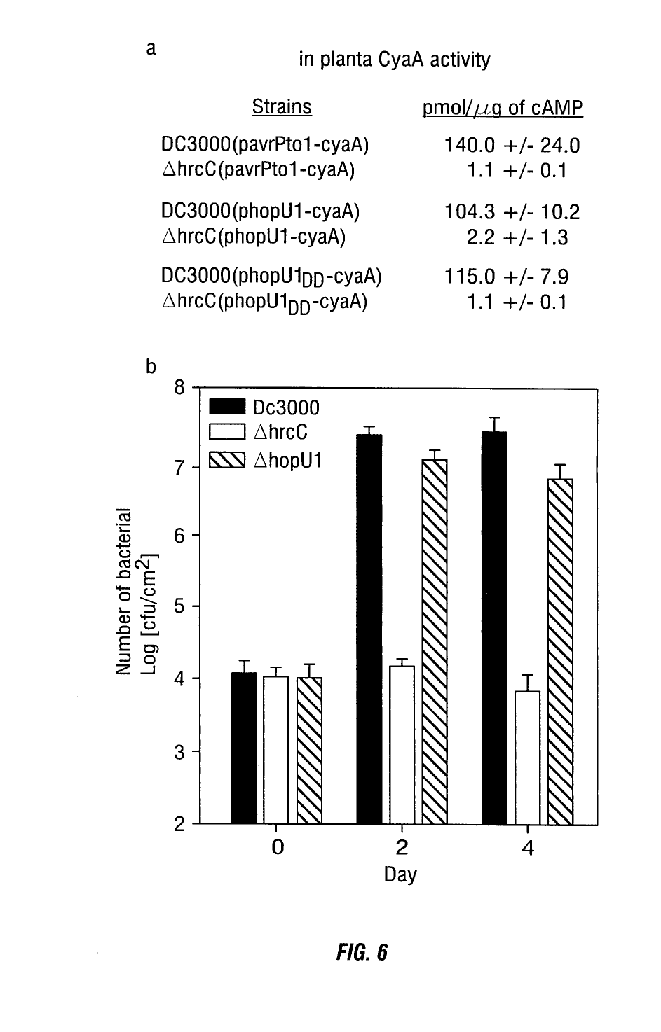

FIG. 7. Ion leakage conductivity assays to quantify cell death in the presence of HopU1. (a) DC3000, the ΔhopU1 mutant, the ΔhopU1 mutant expressing hopU1 in trans (phopU1) were infiltrated into tobacco leaves at threshold cell densities and after 24 h conductivity assays were performed on leaf tissue. The ΔhopU1 mutant infiltrated leaf tissue had greater conductivity than leaf tissue infiltrated with wild type DC3000 consistent with HopU1 acting as an HR suppressor. (b) The DC3000 ΔhopU1 mutant carrying either a vector control (vector), a hopU1 construct (phopU1), or a hopU1 ADP-RT catalytic mutant construct (phopU1_{DD}) were infiltrated into Nicotiana tabacum (tobacco) at threshold cell densities. Conductivity assays were carried out after 24 h. When HopU1_{DD} was expressed the conductivity was similar to the DC3000 ΔhopU1 mutant indicating the HR suppression ability of HopU1 required a functional ADP-RT catalytic site.

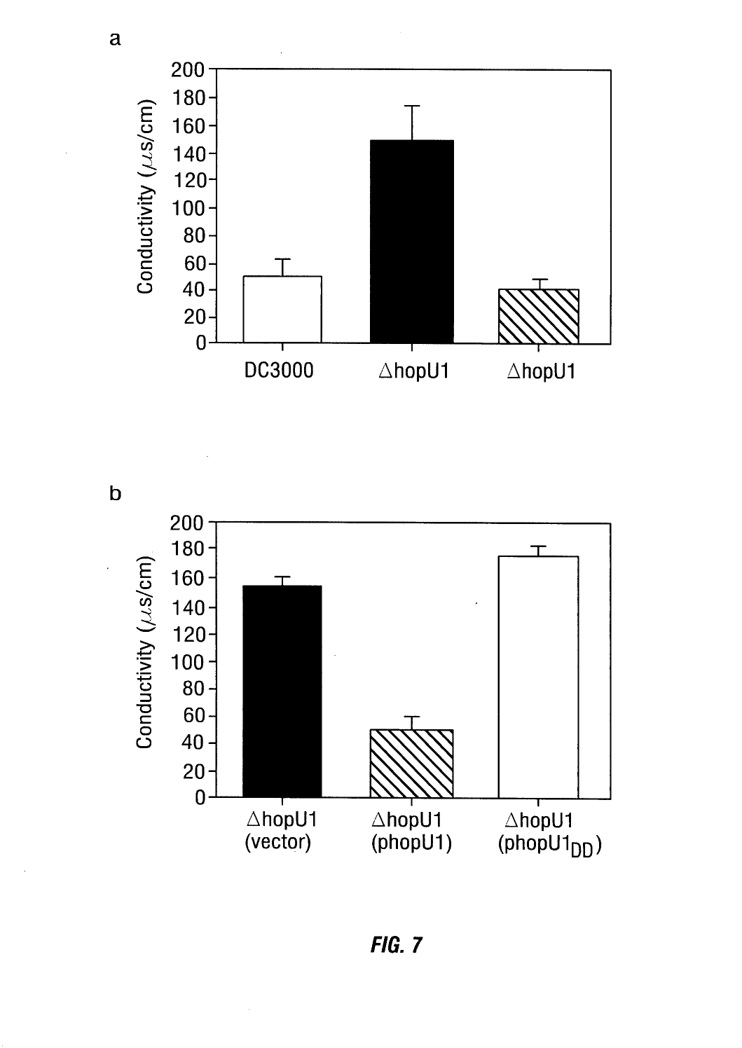

FIG. 8. HopU1-expressing Arabidopsis plants elicit a delayed atypical AvrRpt2-dependent HR. A. thaliana Col-0 plants expressing either HopU1-HA or HopU1_{DD}-HA were infiltrated with DC3000 with or without the type III effector gene avrRpt2. After 20 h plants were assessed for production of an HR. An HR typically develops in HopU1-HA plants at about 25 h, approximately 10 h longer than it takes to develop in wild type plants.

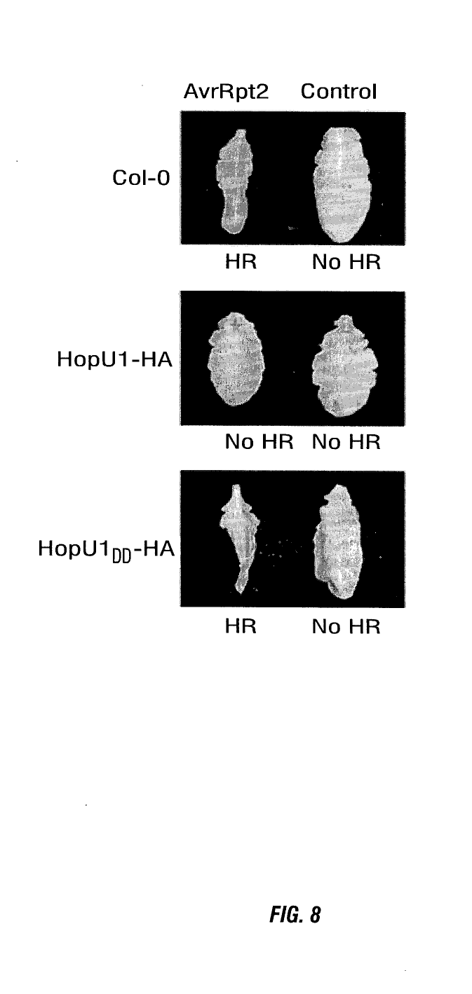

FIG. 9. Representative examples of identification of protein spots that migrated similarly to ADP-RT activity spots. (a) An Arabidopsis soluble protein extract was incubated with HopU1-His in the presence of [³²P]-NAD. The reaction mix was subjected to two-dimensional SDS-PAGE and stained with Coomassie blue (top panel). The dried polyacrylamide gel was exposed to autoradiography film (bottom panel). The black arrowhead in the top panel identifies a protein that possessed the same migration as an ADP-ribosylated activity spot in the bottom panel (black arrowhead). (b) As described in the Methods, a crude A. thaliana Col-0 extract was separated on a HighTrap Q anion exchange column and fractions were examined for proteins that were ADP-ribosylated by HopU1-His. ADP-ribosyltransferase assays were carried out on all fractions, the reaction mixtures were subjected to SDS-PAGE, and autoradiography. Autoradiograms from the fractions that contained the majority of HopU1-His substrates based on a high level of ³²P incorporation are shown. (c, d) A. thaliana extract fractions after ion exchange chromatography were used in ADP-ribosyltransferase assays and subjected to two-dimensional SDS-PAGE. Shown are representative gels (top panel in both c- and d) stained with Coomassie blue and an autoradiogram (lower panel in both c-d) containing aliquots from fraction 42 (panel c) or 23 (panel d). The black arrowheads present in the top panels identify protein spots that migrated similarly with protein spots that were modified by HopU1His in the lower panel. Molecular weight markers in kilodaltons are indicated on the left (a-d).

FIG. 10. Localization of HopU1-GUS, HopU1-GFP, and substrate GFP fusions in plant cells. (a) Biolistic transformation of onion epidermal cells was performed with Agrobacterium binary constructs carrying either the uidA gene encoding β-glucuronidase (GUS) or the uidA gene fused to HopU1 (HopU1-GUS). After 24 h the plant tissue was histochemically stained for GUS activity and photographed. (b-d) hopU1-GFP DNA, DNA encoding HopU1 substrate GFP fusions, or GFP encoding DNA was transiently delivered into N. benthamiana leaves with Agrobacterium-mediated delivery. After 40 h, the leaf tissue was viewed with confocal microscopy using single excitation (488 nm) and dual-emission filters (522 nm for GFP and 680 nm for chloroplast autofluorescence). (b) HopU1-GFP or GFP control merged images are shown. (c) GR-RBP-GFP fusions are shown. (d) Chloroplast RNA-binding proteins encoded by the A. thaliana loci At2g37220. 1, At5g50250.1, and At4g24770.1 fused to GFP were visualized for GFP (top panel), and for chloroplast autofluorescence (middle panel). GFP and autofluorescence images were merged in the bottom panel.

FIG. 11. Characterization of a SALK T-DNA knock-out line designated Atgrp7-1 defective in the AtGRP7 gene. (a) Schematic representation of the AtGRP7 locus in A. thaliana Col-0 in relation to the T-DNA insertion in SALK line SALK_{—}039556.21.25.x. Black boxes depict the two exons (E1 and E2) of AtGRP7; the primers used to confirm the homozygosity of the Atgrp7-1 mutant (listed in Table 3 as P2012, P1947, and P2013) are depicted here as P1, P2, and P3; the T-DNA insertion is represented as a triangle. (b) PCR with primer set P1/P3, which was used to amplify wild type Col-0 DNA, and primer set P1/P2, which was used to amplify a junction band containing both A. thaliana DNA and T-DNA confirmed that the Atgrp7-1 mutant is homozygous for the T-DNA insert. (c) Reverse transcriptase (RT)-PCR with primers (P1932 and P1929 in Table 3) that anneal to the full length coding sequence showing that AtGRP7 mRNA was not detectable in the Atgrp7-1 mutant line. RT-PCR reactions contained RNA from wild type Col-0 plants or the Atgrp7-1 mutant; inclusion or exclusion of RT in the reactions is indicated by a ‘+’ or ‘−’. (d) Immunoblot analysis (left panel) of soluble proteins from leaves of A. thaliana Col-0 and Atgrp7-1 mutant plants using anti-GRP antibodies, which recognizes AtGRP7. HopU1-His ADP-RT assays were performed with the same samples. The autoradiogram (right panel) shows the absence of an ADP-RT high-affinity band in the Atgrp7-1 lane (indicated with an arrow).

FIG. 12. Characterization of a SALK T-DNA knock-out line designated Atgrp7-2 defective in the AtGRP7 gene. (a) Schematic representation of the AtGRP7 locus in A. thaliana Col-0 in relation to the T-DNA insertion in SALK line SALK_{—}051743.42.85.x. Black boxes depict the two exons (E1 and E2) of AtGRP7; the primers used to confirm the homozygosity of the Atgrp7-2 mutant (listed in Table 3 as P2456, P1947, and P2457) are depicted here as P4, P2, and P5; the T-DNA insertion is represented as a triangle. (b) PCR with primer set P4/P5, which was used to amplify wild type Col-0 DNA, and primer set P2/P5, which was used to amplify a junction band containing both A. thaliana DNA and T-DNA confirmed that the Atgrp7-2 mutant is homozygous for the T-DNA insert. The junction band was sequenced and the T-DNA insert was found to be within the coding region of AtGRP7 in the first exon. This is a different location than indicated on the SIGnAL website (http://signal.salk.edu/). (c) Reverse transcriptase (RT)-PCR with primers (P1932 and P1929 in Table 3) that anneal to the full length coding sequence showing that AtGRP7 mRNA was not detectable in the Atgrp7-2 mutant line. RT-PCR reactions contained RNA from wild type Col-0 plants or the Atgrp7-2 mutant. (d) Immunoblot analysis of soluble proteins from leaves of A. thaliana Col-0 and Atgrp7-2 mutant plants using anti-GRP antibodies, which recognizes AtGRP7. (e) Bacterial growth assays with wild type P. s. tomato DC3000 and the ΔhrcC mutant on wild type A. thaliana Col-0 plants compared to Atgrp7-2 mutant plants. (f) Disease symptoms on wild type A. thaliana Col-0 and Atgrp7-2 plants infected with DC3000. Pictures were taken 5 days after spray-inoculation. (g) Callose deposition in A. thaliana Col-0 and Atgrp7-2 mutant plants 16 h after treatment with flg22. The number of callose foci per field of view for 20 leaf regions (4 fields of view from 5 different leaves) were averaged and the standard errors are indicated as error bars.

FIG. 13. Proposed model of suppression of plant innate immunity by the HopU1 ADP-RT type III effector. P. syringae pv. tomato DC3000 injects HopU1 and other type III effectors into plant cells via its type III secretion system. Innate immunity can be triggered by a type III effector (depicted above as the ‘A’-labelled diamond) if it is recognized directly or indirectly by a resistance (R) protein or by a PAMP perceived by PAMP receptors (e.g., FLS2). Activated type III effector- and PAMP-triggered signal transduction pathways result in transcriptional changes in the plant that favor plant resistance to the pathogen. Our model predicts that in the absence of HopU1 (U1) (as in 1), innate immunity-induced mRNAs are stabilized, transported, or processed by RNA-binding proteins (RBPs) in the nucleus or the cytoplasm (as shown) resulting in the production of immunity-related products that facilitate the outputs of innate immunity, including the deposition of callose and the HR. In the presence of HopU1 (as in 2), one or several RBPs are ADP-ribosylated by HopU1 altering their normal function and, subsequently, affecting RNA metabolism. For example, the ADP-ribose modification may prevent a RBP from binding mRNA as depicted. This results in a suppressed innate immune response favoring the survival of the pathogen. cw, cell wall; pm, plasma membrane; nm, nuclear membrane.

FIG. 14. HopU1-His ADP-ribosylates at least one protein in human cancer cell extracts. ADP-RT reactions (using [³²P]-NAD) were carried out with 34 μg extracts of 22Rv1 human cancer cells and 200 ng of HopU1-His or HopU1_{DD}-His. Each reaction was separated with SDS-PAGE and exposed to X-ray film for 10 m.

### DETAILED DESCRIPTION OF THE PREFERRED EMBODIMENT

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Overview

The present invention relates to the discovery that a Hrp-dependent outer protein (Hop) HopU1 is an adenosine diphosphate ribosyltransferase (ADP-RT) that acts as a suppressor of the innate immune responses of a plant, for example in Nicotiana tabacum cv. Xanthi (tobacco) or Arabidopsis thaliana, exposed to a biotic stress. In vitro studies identified five RNA Binding Protein (RBP) plant proteins that are ADP-ribosylated by HopU1. Without wishing to be bound by this theory, it is believed that expressing HopU1 in a plant suppresses innate immunity in a manner related to HopU1's ability to ADP-ribosylate a substrate, thereby interfering with the substrate's ability to bind RNA and consequently quelling the host's immunity by affecting RNA metabolism and the defense transcriptome.

Modulation of the expression level and activity of HopU1 and its substrates in a plant would provide a mechanism for manipulating the plant's innate immune response. Accordingly, the present invention provides methods for the production of plants with enhanced or suppressed innate immune responses, for example, to a biotic stress, such as a pathogen. In one aspect, the methods include introducing into a plant or an animal cell, tissue, or plant one or more polynucleotides encoding HopU1 polypeptides having the enzymatic activity of ADP-RT. This may be accomplished by introducing HopU1 polynucleotides into the plant nuclear and/or plant plastidial genome.

In one aspect, the methods include introducing into a plant or animal cell, tissue, plant one or more polynucleotides encoding HopU1 substrate polypeptides capable of being ADP-ribosylated by HopU1. This may be accomplished by introducing the HopU1 substrate polynucleotide into the plant nuclear and/or plant plastidial genome.

Plants expressing HopU1 will provide suppressed innate immune response to a biotic stress. Additionally, they serve as a system in which to determine the role of targets of HopU1 and other components in a plant's innate immune response. Advantageously, plants over-expressing a HopU1 substrate will provide an enhanced innate immune response to the biotic stress.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5^{th} ed., Prentice-Hall; Dhringra and Sinclair, (1985) Basic Plant Pathology Methods, CRC Press; Maniatis, et al., (1982) Molecular Cloning: A Laboratory Manual; DNA Cloning, vols. I and II, Glover, ed. (1985); Oligonucleotide Synthesis, Gait, ed. (1984); Nucleic Acid Hybridization, Hames and Higgins, eds. (1984); and the series Methods in Enzymology, Colowick and Kaplan, eds, Academic Press, Inc., San Diego, Calif.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

Suitable plants for use in methods of the present invention include but are not limited to, an almond, apple, apricot, Arabidopsis, artichoke, atriplex, avocado, barley, beet, birch, brassica, cabbage, cacao, cantaloupe, carnations, castorbean, cauliflower, celery, clover, coffee, corn, cotton, cucumber, garlic, grape, grapefruit, hemp, hops, lettuce, maple, melon, mustard, oak, oat, olive, onion, orange, pea, peach, pear, pepper, pine, plum, poplar, potato, prune, radish, rape, rice, roses, rye, salicornia, sorghum, soybean, spinach, squash, strawberries, sunflower, sweet corn, tobacco, tomato or wheat plant. In another aspect, the term animal includes, but is not limited to, a mouse, cow, pig, dog, cat, monkey or human.

As used herein, the term “plant” includes reference to an immature or mature whole plant, including a plant that has been detasseled or from which seed or grain has been removed. Seed or embryo that will produce the plant is also considered to be the plant.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used interchangeably herein, a “HopU1 activity”, “biological activity of HopU1” or “functional activity of HopU1”, refers to an activity exerted by a HopU1 protein, polypeptide or portion thereof as determined in vivo, or in vitro, according to standard techniques. For example, HopU1 activity includes ADP-ribosylating a substrate in vitro or in vivo, or acting to suppress the innate immune response of a plant as provided by the present invention.

As used herein, the term HopU1 includes but is not limited to the sequence of Pseudomonas syringae pv. tomato str. DC3000 disclosed in GenBank accession no. AA054045.1 GI:28850966 (protein) and AE016853 (nucleotide). (Buell, R et al. The complete genome sequence of the Arabidopsis and tomato pathogen Pseudomonas syringae pv. tomato DC3000, Proc. Natl. Acad. Sci. U.S.A. 100(18):10181-10186 (2003), and also includes conservatively modified variants, regardless of source and any other variants which retain the biological properties of the HopU1, for example, HopU1 activity. The term “HopU1 polypeptide” refers to one or more amino acid sequences. The term is also inclusive of fragments, variants, homologs, alleles or precursors (e.g., preproproteins or proproteins) thereof. A “HopU1 protein” comprises a HopU1 polypeptide. Unless otherwise stated, the term “HopU1 nucleic acid” means a nucleic acid comprising a polynucleotide (“HopU1 polynucleotide”) encoding a HopU1 polypeptide.

The term “HopU1 substrate” as used herein refers to a molecule ADP-ribosylated by a HopU1 protein or polypeptide. The term “HopU1 substrate polypeptide” refers to one or more amino acid sequences. The term is also inclusive of fragments, variants, homologs, alleles or precursors (e.g., preproproteins or proproteins) thereof. A “HopU1 substrate protein” comprises a HopU1 substrate polypeptide. Unless otherwise stated, the term “HopU1 substrate nucleic acid” means a nucleic acid comprising a polynucleotide (“HopU1 substrate polynucleotide”) encoding a HopU1 substrate polypeptide. Exemplary substrates include RBPs, such as GR-RBPs and CP-RBPs. HopU1 substrate polypeptides that are targets of HopU1 suppress the innate immune response of a plant when ADP-ribosylated by HopU1 or enhance the innate immune system when over-expressed in a plant cell or plant. It is also contemplated that certain residues of the HopU1 substrate may be altered to prevent ADP-ribosylation by HopU1.

The term “suppressing the innate immune response” as used herein includes making a plant's immune system more susceptible to a biotic stress for example, pathogens, such as bacteria, fungi or viruses, including microbial invasions. Suppressing the innate immune response includes decreasing the plant's innate immune response, for example, so that the HR of the plant is decreased or inhibited upon exposure to a biotic stress. The suppression of the innate immune response of a subject plant can be determined using a variety of in vitro or in vivo measurements of innate immune responses to a biotic stress, for example, including the levels of reactive oxygenation species (ROS) and nitric oxide (NO), changes in cytoplasmic Ca2+ levels, production of pathogen-related (PR) gene expression, phytoalexin production, papillae formation (i.e thickening of the cell wall), and callose (P-1-3 glucan deposition) in the cell wall. (Dangl, J. L., and J. D. Jones. Plant pathogens and integrated defence responses to infection. Nature 411:826-833 (2001); Gomez-Gomez, L., and T. Boller. Flagellin perception: a paradigm for innate immunity. Trends Plant Sci. 7:251-256 (2002); Heath, M. C. Nonhost resistance and nonspecific plant defenses. Curr. Opin. Plant Biol. 0.3:315-319 (2000); and Jones, D. A., and D. Takemoto. Plant innate immunity—direct and indirect recognition of general and specific pathogen-associated molecules. Curr. Opin. Immunol. 16:48-62 (2004)), or the hypersensitive response, present in the plant. (Goodman, R. N., and A. J. Novacky. The hypersensitive reaction of plants to pathogens: A resistance phenomenon. APS Press, St. Paul (1994); and Heath, M. C. Hypersensitive response-related death. Plant Mol. Biol. 44:321-33 (2000)).

The term “enhancing the innate immune response” as used herein includes making a plant's immune system more resistant to a biotic stress for example, pathogens, such as bacteria, fungus or viruses, including microbial invasions. Enhancing the innate immune response includes increasing the plant's innate immune response, for example, so that the HR of the plant is increased or maintained upon exposure to a biotic stress. The enhancement of the innate immune response of a subject plant can be determined using a variety of in vitro or in vivo measurements of innate immune responses to a biotic stress, for example, including the levels of reactive oxygenation species (ROS) and nitric oxide (NO), changes in cytoplasmic Ca2+ levels, production of pathogen-related (PR) gene expression, phytoalexin production, papillae formation (i.e thickening of the cell wall), and callose (β-1-3 glucan deposition) in the cell wall. (Dangl, J. L., and J. D. Jones. Plant pathogens and integrated defence responses to infection. Nature 411:826-833 (2001); Gomez-Gomez, L., and T. Boller. Flagellin perception: a paradigm for innate immunity. Trends Plant Sci. 7:251-256 (2002); Heath, M. C. Nonhost resistance and nonspecific plant defenses. Curr. Opin. Plant Biol. 0.3:315-319 (2000); and Jones, D. A., and D. Takemoto. Plant innate immunity—direct and indirect recognition of general and specific pathogen-associated molecules. Curr. Opin. Immunol. 16:48-62 (2004)), or the hypersensitive response, present in the plant. (Goodman, R. N., and A. J. Novacky. The hypersensitive reaction of plants to pathogens: A resistance phenomenon. APS Press, St. Paul (1994); and Heath, M. C. Hypersensitive response-related death. Plant Mol. Biol. 44:321-33 (2000)).

The term “hypersensitive response (HR)” refers to a rapid, defense-associated programmed death of plant cells in contact with the pathogen (Alfano & Collmer, J. Bacteriol. 179:5655-5662 (1997)).

The term “innate immune response” includes any response of the immune system including both non-specific and specific responses.

ADP-ribosylation, as used herein, is a chemical or biochemical reaction involving the addition of ADP to a substrate, to form a ribosylated substrate or product.

As used herein, the term “biotic stress” refers to any pathogen, such as a bacteria, fungi, protozoa and viruses or any biological organism that is harmful to plants and includes, an organism or an infectious agent whose infection around or inside the cells of viable plant tissue elicits a disease response. Other examples of biotic stresses include those injuries resulting from infections by insects, nematodes, snails, mites, weeds, and physical damage caused by people (i.e. grazing, tredding, etc.).

A “subject plant or plant cell” is one in which genetic alteration, such as transformation, has been effected as to a gene of interest, or is a plant or plant cell which is descended from a plant cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce the innate immune response of the plant to a biotic stress; or (e) the subject plant or plant cell itself, under conditions in which the gene of interest (HopU1 or HopU1 substrate) is not expressed.

I. Method of Suppressing the Innate Immune Response of a Plant.

To date, to the inventor's knowledge, ADP-RTs have not been implicated in the suppression of the innate immune response. Furthermore, genes that encode ADP-RTs have been found in eukaryotes but have not been identified in plants (Corda, D. & Di Girolamo, M. Functional aspects of protein mono-ADP-ribosylation. EMBO 22, 1953-1958 (2003)). In one aspect, the present invention provides a method for suppressing the innate immune response of a plant. In one aspect, the method includes expressing or over-expressing a polynucleotide that encodes a HopU1 polypeptide with HopU1 activity in a prokaryotic or eukaryotic cell, for example, a bacterial, plant or animal cell. As used herein, the term “over-express” or “over-expressing” refers to the production of a HopU1 polypeptide in a plant cell in amounts exceeding that normally produced in the plant or animal cell.

Polypeptides of HopU1 preferably possess one or more of the bioactivities of the HopU1 protein, including ADP-ribosylating a substrate in vitro or in vivo, acting to suppress the innate immune response of a plant as provided by the present invention. Also, included for use in the present invention are HopU1 substrate polypeptides that are targets of HopU1 that suppress the innate immune response of a plant when ADP-ribosylated by HopU1 or enhance the innate immune system when over-expressed in a plant cell or plant. It is also contemplated that certain residues of the HopU1 substrate may be altered to prevent ADP-ribosylation by HopU1.

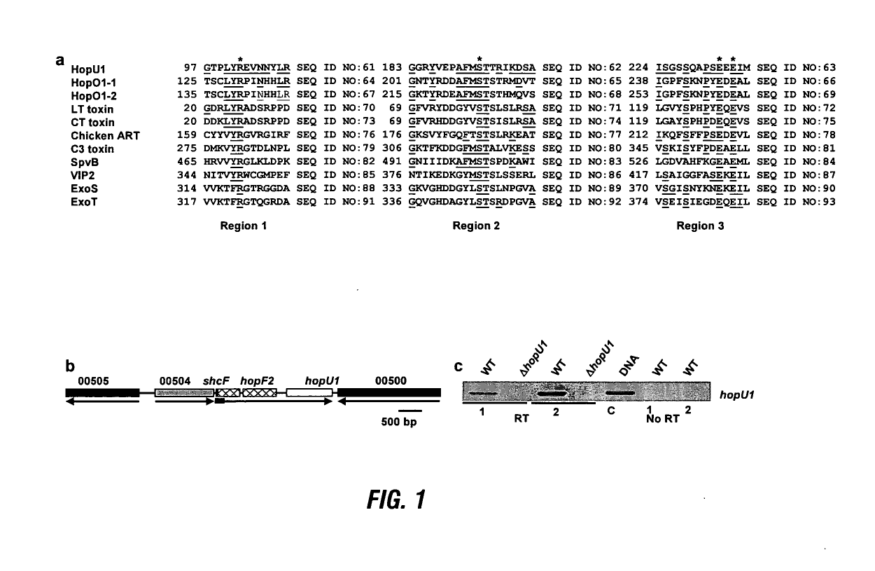

In one aspect of the invention, the protein may be a HopU1 polypeptide, a variant HopU1 polypeptide and/or have additional amino acid molecules, such as amino acid residues (contiguous encoded sequence) that are naturally associated with it or heterologous amino acid residues/peptide sequences. Such a protein can have a few additional amino acid residues or can comprise several hundred or more additional amino acids. Fragments of the HopU1 polypeptide, for example, a fragment that comprises at least 8 or more contiguous amino acid residues from a HopU1 polypeptide or variant protein as described herein, that retain one or more of the biological activities of a HopU1 polypeptide, for example, ADP-ribosylating a substrate in vitro or in vivo, and/or acting to suppress the innate immune response of a plant or animal as provided by the present invention. Also included, therefore, are variants or fragments of the HopU1 polypeptide that encodes a polypeptide having a ADP-RT active (catalytic) site, in the conserved region 3 with glutamic acids at positions 233 and 235 (FIG. 1a), that correlates with ribosylation activity of a HopU1 substrate. Such a domain can be identified on the basis of sequence similarity with HopU1 ADP-RT domain and other proteins with ADP-RT domains (e.g., Cholera toxin), especially over critical conserved residues. Additional HopU1 polypeptides, variants or fragments may include those with substitutions, deletions, or insertions, as compared to a known sequence, for example HopU1, including those that alter HopU1 activity.

The polynucleotides encoding polypeptides with HopU1 activity can be prepared by any suitable technique in the art, including synthesis or amplification as described in example 3. As described above, HopU1 polypeptides may be variants or fragments of HopU1. Any of these can be attached to heterologous sequences to form chimeric or fusion proteins for use in methods of the present invention. Such chimeric and fusion proteins comprise a Hop1 polypeptide operatively linked to a heterologous protein having an amino acid sequence not substantially homologous to the variant protein. “Operatively linked” indicates that the variant protein and the heterologous protein are fused in-frame. The heterologous protein can be fused to the N-terminus or C-terminus of the HopU1 polypeptide, variant or fragment of HopU1.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present invention also encompass derivatives or analogs in which a substituted amino acid residue is not one encoded by the genetic code, in which a substitute group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

The polynucleotides encoding polypeptides with HopU1 activity can be obtained using methods that are known to those of skill in the art. Suitable nucleic acids (e.g., cDNA, genomic, microRNAs or RNAi, or subsequences) can be cloned, or amplified by in vitro methods such as the polymerase chain reaction (PCR) using suitable primers, the ligase chain reaction (LCR), the transcription-based amplification system (TAS), the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C&EN 36-47; The Journal Of NIH Research (1991) 3:81-94; (Kwoh et al. (1989) Proc. Natl. Acad. Sci. USA 86:1173; Guatelli et al. (1990) Proc. Natl. Acad. Sci. USA 87:1874; Lomell et al. (1989) J. Clin. Chem., 35:1826; Landegren et al., (1988) Science 241:1077-1080; Van Brunt (1990) Biotechnology 8:291-294; Wu and Wallace (1989) Gene 4:560; and Barringer et al. (1990) Gene 89:117.

One of skill would recognize that modifications could be made to a HopU1 or HopU1 substrate polypeptide or protein without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Modifications to the HopU1 or HopU1 substrate polypeptide or protein also include but are not limited to conservative amino acid substitutions and those that are 0phenotypically silent. Conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr and Trp. Guidance concerning which amino acid changes are likely to be phenotypically silent is found in Bowie et al., Science 247:1306 1310 (1990).

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119. A polynucleotide encoding a polypeptide with HopU1 activity or a HopU1 substrate may be introduced into either a prokaryotic or eukaryotic plant or animal host cell through the use of a vector, plasmid or construct, or TTSS, such as P. syringae or P. aeruginosa. An expression cassette may contain a promoter operably linked to a nucleic acid that encodes the polypeptide. Cells are transfected with an expression vector for the polynucleotide encoding a polypeptide with HopU1 activity or a HopU1 substrate, or variant or fragment thereof, and if desired the effect on ADP ribosylation may be measured by a suitable method, such as the assays described herein. The expression cassettes are typically included on expression vectors that are introduced into suitable host cells, including for example, bacterial, insect, fungal, plant or animal cells. For example, plant expression vectors may include a cloned plant gene under the transcriptional control of 5′ and 3′ regulatory sequences and a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

The expression vectors of the invention can be transferred into the chosen eukaryotic host cell by methods known to those of ordinary skill in the art including, for example, calcium phosphate transfection, DEAE-dextran mediated transfection, transvection, microinjection, cationic lipid-mediated transfection, electroporation, transduction, scrape loading, ballistic introduction, infection, Methods To Get Into Plant Cells or other methods. (See Molecule Cloning: A Laboratory Manual, 2^{nd} ed., Vol. 1-3, ed. Sambrook et al., Cold Spring Harbor Laboratory Press (1989)). Transformed cells can be selected, for example, by resistance to antibiotics conferred by genes contained on the plasmids, such as the amp, gpt, neo and hyg genes.

Numerous methods for introducing foreign genes into plants are known and can be used to insert a HopU1 or HopU1 substrate polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., Science 227:1229-31 (1985)), electroporation, micro-injection, and biolistic bombardment.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 5:320-335), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,850), direct gene transfer (Paszkowski et al. (1985) EMBO J. 3:2717-2722), and ballistic particle acceleration (see, for example, Sanford et al., U.S. Pat. No. 5,955,050; Tomes et al., U.S. Pat. No. 5,879,918; Tomes et al., U.S. Pat. No. 5,886,255; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO00/28058). Also see Weissinger et al. (1988) Ann. Rev. Genet. 22:521-577; Sanford et al. (1987) Particulate Science and Technology 5:27-37 (onion); Christou et al. (1988) Plant Physiol. 87:671-675 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-325 (soybean); Datta et al. (1990) Biotechnology 8:736-750 (rice); Klein et al. (1988) Proc. Natl. Acad. Sci. USA 85:5305-5309 (maize); Klein et al. (1988) Biotechnology 6:559-563 (maize); Tomes, U.S. Pat. No. 5,250,855; Buising et al., U.S. Pat. Nos. 5,322,783 and 5,325,656; Tomes et al. (1995) “Direct DNA Transfer into Intact Plant Cells via Microprojectile Bombardment,” in Plant Cell, Tissue, and Organ Culture Fundamental Methods, ed. Gamborg (Springer-Verlag, Berlin) (maize); Klein et al. (1988) Plant Physiol. 91:550-555 (maize); Fromm et al. (1990) Biotechnology 8:833-839 (maize); Hooykaas-Van Slogteren et al. (1985) Nature (London) 311:763-765; Bowen et al., U.S. Pat. No. 5,736,369 (cereals); Bytebier et al. (1987) Proc. Natl. Acad. Sci. USA 85:5355-5359 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:515-518 and Kaeppler et al. (1992) Theor. Appl. Genet. 85:560-566 (whisker-mediated transformation); D'Halluin et al. (1992) Plant Cell 5:1595-1505 (electroporation); Li et al. (1993) Plant Cell Reports 12:250-255 and Christou and Ford (1995) Annals of Botany 75:507-513 (rice); Osjoda et al. (1996) Nature Biotechnology 15:755-750 (maize via Agrobacterium tumefaciens); Leelavathi et al. (2004) Plant Cell Reports 22:465-470 (cotton via Agrobacterium tumefaciens); Kumar et al. (2004) Plant Molecular Biology 56:203-216 (cotton plastid via bombardment); all of which are herein incorporated by reference.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells such Agrobacterium or Rhizobium. Examples are promoters that preferentially initiate transcription in certain tissues, such as leaves, roots, seeds, fibers, xylem vessels, tracheids, or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light.

Another type of promoter is a developmentally regulated promoter, for example, a promoter that drives expression during pollen development. Examples of pollens-specific promoters include but are not limited to a pollen-specific pectate lyase gene promoter or PG47 gene promoter, an another specific RTS2 gene promoter, SGB6 gene promoter, or G9 gene promoter. Tissue preferred, cell type specific, developmentally regulated, and inducible promoters constitute the class of “non-constitutive” promoters. A “constitutive” promoter is a promoter, which is active under most environmental conditions. Induced expression can also be achieved using chemicals such as estradiol using inducible vector Per8 (Jianru Zuo, Qi-Wen Niu, Nam-Hai Chua (2000) An estrogen receptor-based transactivator XVE mediates highly inducible gene expression in transgenic plants. The Plant Journal 24 (2), 265-273. doi:10.1046/j.1365-313x.2000.00868.x).

A number of promoters can be used in the practice of the invention, including the native promoter of the HopU1 or HopU1 substrate polynucleotide sequence, for example, the promoter of the HopU1 substrate of the crop plant of interest. The promoters can be selected based on the desired outcome. The HopU1 or HopU1 substrate polynucleotide can be combined with constitutive, tissue-preferred, inducible, or other promoters for expression in plants.

A plant promoter or promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30); and maize H3 histone (Lepetit, et al., (1992) Mol. Gen. Genet. 231:276-85; and Atanassvoa, et al., (1992) Plant Journal 2(3):291-300); ALS promoter, as described in PCT Application No. WO 96/30530; and other transcription initiation regions from various plant genes known to those of skill. For the present invention ubiquitin is the preferred promoter for expression in monocot plants.

Tissue-preferred promoters can be utilized to target enhanced type A RR expression within a particular plant tissue. By “tissue-preferred” is intended to mean that expression is predominately in a particular tissue, albeit not necessarily exclusively in that tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen. Genet. 255(3):337-353; Russell et al. (1997) Transgenic Res. 6(2):157-168; Rinehart et al. (1996) Plant Physiol. 112(3):1331-1351; Van Camp et al. (1996) Plant Physiol. 112(2):525-535; Canevascini et al. (1996) Plant Physiol. 112(2):513-525; Yamamoto et al. (1995) Plant Cell Physiol. 35(5):773-778; Lam (1995) Results Probl. Cell Differ. 20:181-196; Orozco et al. (1993) Plant Mol. Biol. 23(6): 1129-1138; Matsuoka et al. (1993) Proc Natl. Acad. Sci. USA 90(20):9586-9590; and Guevara-Garcia et al. (1993) Plant J. 5(3):595-505. Such promoters can be modified, if necessary, for weak expression. See, also, U.S. Patent Application No. 2003/0074698, herein incorporated by reference.

Expression of HopU1 and over-expression of HopU1 substrates may be achieved by any number of methods, including but not limited to transient transfection, integration, gene amplification, plant transformation or any other molecular process which results in amounts of HopU1 polypeptide exceeding those amounts typically found in the host cell as described elsewhere in the application. The typical range of expression in a given cell type can be determined by routine techniques as by assaying the HopU1 protein, its mRNA, or its gene.

The expression level of the HopU1 or HopU1 substrate polypeptide may be measured directly, for example, by assaying for the level of the HopU1 or HopU1 substrate polypeptide in the plant, or indirectly, for example, by measuring the HopU1 activity of the HopU1 or HopU1 substrate polypeptide in the plant. Methods for determining the HopU1 or HopU1 substrate expression or activity may be determined using standard techniques or assays such as immunoassay, PAGE, western blot, Southern and Northern blots, and competition hybridization procedures and Y, including the evaluation of ADP-RT activity in vitro or in vivo in various expression systems.

Functional assays for ADP-ribosyltransferase can be conducted by incubating the HopU1 polypeptide with a target protein (such as the poly-L-arginine (see Example 7)), or a known substrate of HopU1, such as AtGRP7 and AtGRP8 (See Table 1) in the presence of nicotinamide adenine dinucleotide (NAD⁺), or an analog labeled with a radioisotope such as ³² P or ³³ P, biotin, or a fluorescent group. (186) ADP-ribosylation can be monitored by incorporation of the label into the protein phase, by a change of size of the target protein (measurable, for example on a protein gel), or by detection of ADP ribose polymers on the target (for example, using commercially available antibody specific for ADP-ribose polymers, or by physical-chemical mechanisms, such as mass spectrometry). Known ADP-RT inhibitors like novobiocin can be used to verify the specificity of the assay. Polypeptides with confirmed ADP-RT activity can then be used in methods of the present invention, for example, suppressing the innate immune system of a plant.

The evaluation of HopU1 expression may also include evaluation of phenotypic changes, such as suppressed immune response in a plant subjected to a biotic stress. Examples of phenotypic changes include but are not limited to the transfer of ADP-ribose of β-NAD onto a HopU1 substrate, HR and other outputs of innate immunity as described herein. As stated above, suppression of the plant's innate immune response may be achieved through expression of HopU1. Thus, modulation of HopU1 expression or activity in a plant or plant cell provides a novel strategy for suppressing the immune system of a plant exposed to a biotic stress.

Methods are provided to reduce or eliminate the level and/or the activity of a HopU1 substrate polypeptide by transforming a plant cell with an expression cassette that expresses a polynucleotide that inhibits the expression of the HopU1 substrate polypeptide. As used herein, the term “under-express”, “under-expression” or “under-expressing” refers to the production of a HopU1 substrate polypeptide in a plant cell in amounts less than that normally produced in the plant or animal cell.

The polynucleotide may inhibit the expression of one or more HopU1 substrate polypeptides directly, by preventing translation of the HopU1 substrate messenger RNA, or indirectly, by encoding a polypeptide that inhibits the transcription or translation of a plant gene encoding a HopU1 substrate polypeptide. Methods for inhibiting or eliminating the expression of a gene in a plant are well known in the art, and any such method may be used in the present invention to inhibit the expression of one or more HopU1 substrate polypeptide.

In accordance with the present invention, the expression of a HopU1 substrate polypeptide is under-expressed, decreased or inhibited if the protein level of the HopU1 substrate polypeptide is statistically significantly lower than the protein level of the same HopU1 substrate polypeptide in a plant that has not been genetically modified or mutagenized to inhibit the expression of that protein. In particular embodiments of the invention, the protein level of the HopU1 substrate polypeptide in a modified plant according to the invention is less than 96%, less than 90%, less than 80%, less than 75%, less than 60%, less than 50%, less than 40%, less than 30%, less than 20%, less than 10%, or less than 5% of the protein level of the same HopU1 substrate polypeptide in a plant that is not a mutant or that has not been genetically modified to inhibit the expression of that HopU1 substrate polypeptide. The expression level of the HopU1 substrate polypeptide may be measured directly, for example, by assaying for the level of HopU1 substrate polypeptide expressed in the plant cell or plant, or indirectly, for example, by measuring the activity of the HopU1 substrate polypeptide in the plant cell or plant. Methods for determining the HopU1 substrate activity of HopU1 substrate polypeptide are described elsewhere herein.

In other embodiments of the invention, the activity of one or more HopU1 substrate is reduced or eliminated by transforming a plant cell with an expression cassette comprising a polynucleotide encoding a polypeptide that inhibits the activity of one or more HopU1 substrates. The HopU1 substrate activity may be inhibited according to the present invention if the HopU1 substrate activity of the genetically modified plant is statistically significantly lower than the activity of the same HopU1 substrate in a plant that has not been genetically modified to inhibit the HopU1 substrate activity. In particular embodiments of the invention, the HopU1 substrate activity of the HopU1 substrate in a modified plant according to the invention is less than 95%, less than 90%, less than 80%, less than 70%, less than 60%, less than 50%, less than 40%, less than 30%, less than 20%, less than 10%, or less than 5% of the HopU1 substrate activity of the same HopU1 substrate in a plant that that has not been genetically modified to inhibit the expression of that HopU1 substrate. The HopU1 substrate activity of a HopU1 substrate is “eliminated” according to the invention when it is not detectable by the assay methods described elsewhere herein. Methods of determining the HopU1 substrate activity of a HopU1 substrate are described elsewhere herein.

In other embodiments, the activity of a HopU1 substrate may be reduced or eliminated by disrupting the gene encoding the HopU1 substrate. The invention encompasses mutagenized plants that carry mutations in HopU1 substrate genes, where the mutations reduce expression of the HopU1 substrate gene or inhibit the HopU1 substrate activity of the encoded HopU1 substrate.

Thus, many methods may be used to reduce or eliminate the activity of a HopU1 substrate. More than one method may be used to reduce the activity of a single HopU1 substrate. In addition, combinations of methods may be employed to reduce or eliminate the activity of two or more different HopU1 substrate polypeptides.

Non-limiting examples of methods of reducing or eliminating the expression of a HopU1 substrate are given below.

In some embodiments of the present invention, a plant cell is transformed with an expression cassette that is capable of expressing a polynucleotide that inhibits the expression of HopU1 substrate polypeptides. The term “expression” as used herein refers to the biosynthesis of a gene product, including the transcription and/or translation of said gene product. For example, for the purposes of the present invention, an expression cassette capable of expressing a polynucleotide that inhibits the expression of at least one HopU1 substrate polypeptide is an expression cassette capable of producing an RNA molecule that inhibits the transcription and/or translation of at least one HopU1 substrate polypeptide. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

In some embodiments of the invention, inhibition of the expression of a HopU1 substrate polypeptide may be obtained by sense suppression or cosuppression. For cosuppression, an expression cassette is designed to express an RNA molecule corresponding to all or part of a messenger RNA encoding a HopU1 substrate polypeptide in the “sense” orientation. Over-expression of the RNA molecule can result in reduced expression of the native gene. Accordingly, multiple plant lines transformed with the cosuppression expression cassette are screened to identify those that show the greatest inhibition of HopU1 substrate polypeptide expression.

The polynucleotide used for cosuppression may correspond to all or part of the sequence encoding the HopU1 substrate polypeptide, all or part of the 5′ and/or 3′ untranslated region of a HopU1 substrate transcript, or all or part of both the coding sequence and the untranslated regions of a transcript encoding HopU1 substrate polypeptide. In some embodiments where the polynucleotide comprises all or part of the coding region for the HopU1 substrate polypeptide, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin et al. (2002) Plant Cell 15:1517-1532. Cosuppression may also be used to inhibit the expression of multiple proteins in the same plant. See, for example, U.S. Pat. No. 5,952,657. Methods for using cosuppression to inhibit the expression of endogenous genes in plants are described in Flavell et al. (1995) Proc. Natl. Acad. Sci. USA 91:3590-3596; Jorgensen et al. (1996) Plant Mol. Biol. 31:957-973; Johansen and Carrington (2001) Plant Physiol. 126:930-938; Broin et al. (2002) Plant Cell 15:1517-1532; Stoutjesdijk et al (2002) Plant Physiol. 129:1723-1731; Yu et al. (2003) Phytochemistry 63:753-763; and U.S. Pat. Nos. 5,035,323, 5,283,185, and 5,952,657; each of which is herein incorporated by reference. The efficiency of cosuppression may be increased by including a poly-dT region in the expression cassette at a position 3′ to the sense sequence and 5′ of the polyadenylation signal. See, U.S. Patent Publication No. 20020058815, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,185 and 5,035,323; herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs which can interact with the homologous promoter region may trigger degradation or methylation to result in silencing. (Aufsatz et al. (2002) PNAS 99 (Suppl. 4):16499-16506; Mette et al. (2000) EMBO J 19(19):5194-5201) See also U.S. Patent Publication 2005/0246796.

In some embodiments of the invention, inhibition of the expression of the HopU1 substrate polypeptide may be obtained by antisense suppression. For antisense suppression, the expression cassette is designed to express an RNA molecule complementary to all or part of a messenger RNA encoding the HopU1 substrate polypeptide. Over-expression of the antisense RNA molecule can result in reduced expression of the native gene. Accordingly, multiple plant lines transformed with the antisense suppression expression cassette are screened to identify those that show the greatest inhibition of HopU1 substrate polypeptide expression.

The polynucleotide for use in antisense suppression may correspond to all or part of the complement of the sequence encoding the HopU1 substrate polypeptide, all or part of the complement of the 5′ and/or 3′ untranslated region of the HopU1 substrate polypeptide transcript, or all or part of the complement of both the coding sequence and the untranslated regions of a transcript encoding the HopU1 substrate polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,952,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu et al (2002) Plant Physiol. 129:1732-1753 and U.S. Pat. Nos. 5,759,829 and 5,952,657. Efficiency of antisense suppression may be increased by including a poly-dT region in the expression cassette at a position 3′ to the antisense sequence and 5′ of the polyadenylation signal. See U.S. Patent Publication No. 2002/0058815.

In some embodiments of the invention, inhibition of the expression of a HopU1 substrate polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of HopU1 substrate polypeptide expression. Methods for using dsRNA interference to inhibit the expression of endogenous plant genes are described in Waterhouse et al. (1998) Proc. Natl. Acad. Sci. USA 95:13959-13965, Liu et al. (2002) Plant Physiol. 129:1732-1753, and WO 99/59029, WO 99/53050, WO 99/61631, and WO 00/59035.

In some embodiments of the invention, inhibition of the expression of one or more type A RR polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Genet. 5:29-38 and the references cited therein.

For hpRNA interference, the expression cassette is designed to express a RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:5985-5990; Stoutjesdijk et al. (2002) Plant Physiol. 129:1723-1731; and Waterhouse and Helliwell (2003) Nat. Rev. Genet. 5:29-38. Methods for using hpRNA interference to inhibit or silence the expression of genes are described, for example, in Chuang and Meyerowitz (2000) Proc. Natl. Acad. Sci. USA 97:5985-5990; Stoutjesdijk et al. (2002) Plant Physiol. 129:1723-1731; Waterhouse and Helliwell (2003) Nat. Rev. Genet. 5:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-150.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 507:319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 507:319-320; Wesley et al. (2001) Plant J. 27:581-590; Wang and Waterhouse (2001) Curr. Opin. Plant Biol. 5:156-150; Waterhouse and Helliwell (2003) Nat. Rev. Genet. 5:29-38; Helliwell and Waterhouse (2003) Methods 30:289-295, and U.S. Patent Publication No. 20030180955.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00905.

Suppression or enhancement of the immune response of a plant to a biotic stress may be determined or inferred from evaluating outputs of innate immunity, using any number of methods including those as described herein. See Example 7. Once animal or plant immune systems perceive biotic stresses, such as PAMPs or AVR proteins or other viral, fungal, and bacterial pathogens, signal transduction pathways are triggered and defense-related outputs are deployed. Examples of physiological responses that occur in plants after activation of innate immunity include the generation of reactive oxygenation species (ROS) and nitric oxide (NO), changes in cytoplasmic Ca2+ levels, production of pathogen-related (PR) gene expression, phytoalexin production, papillae formation (i.e thickening of the cell wall), and callose (β-1-3 glucan deposition) in the cell wall. (Dangl, J. L., and J. D. Jones. Plant pathogens and integrated defence responses to infection. Nature 411:826-833 (2001); Gomez-Gomez, L., and T. Boller. Flagellin perception: a paradigm for innate immunity. Trends Plant Sci. 7:251-256 (2002); Heath, M. C. Nonhost resistance and nonspecific plant defenses. Curr. Opin. Plant Biol. 0.3:315-319 (2000); and Jones, D. A., and D. Takemoto. Plant innate immunity—direct and indirect recognition of general and specific pathogen-associated molecules. Curr. Opin. Immunol. 16:48-62 (2004)), or the hypersensitive response, a programmed cell death response similar to apoptosis in. (Goodman, R. N., and A. J. Novacky. The hypersensitive reaction of plants to pathogens: A resistance phenomenon. APS Press, St. Paul (1994); and Heath, M. C. Hypersensitive response-related death. Plant Mol. Biol. 44:321-33 (2000)).

Plants expressing or over-expressing HopU1 and/or those with under-expressed or decreased levels of one or more HopU1 substrates are exposed to or challenged with a biotic stress, for example, viral, fungal, and bacterial pathogens such as P. syringae tomato DC3000, Oomycete pathogen Peronospora parasitica (that causes Downy mildew), of flg22 and the suppression of the innate immune system determined. Suppression of the innate immune system of a plant by HopU1 may be determined by evaluating callose deposition in a plant cell wall in a plant exposed to a biotic stress. Callose may be easily stained with a fluorescent dye aniline blue and levels assessed accordingly. (Adam, L., and S. C. Somerville. Genetic characterization of five powdery mildew disease resistance loci in Arabidopsis thaliana Plant J. 9:341-356 (1996)). Plants expressing or over-expressing HopU1, for example, HopU1 transgenic plants, will produce reduced amounts of callose, for example, significantly reduced amounts of callose, as compared to a control plant. The control plant may be a plant that does not have the expression or over-expression of HopU1 or one that is only exposed to a biotic stress.

Another way in which to evaluate the suppression of the innate immune system of a plant is through the production of reactive oxygen species (ROS). (Mittler, R. Oxidative stress, antioxidants and stress tolerance. Trends Plant Sci. 7:405-410 (2002)). Levels of ROS may be determined in plants or exposed to a biotic stress using any suitable technique, including but not limited to nitroblue tetrazolium (NBT) based assays or fluoroscent probe dichlorohydrofluorescein diacetate (DCF-DA). NBT is a dye that form an insoluble blue precipitate in the presence of ROS that can be easily viewed. Leaves of a plant expressing or over-expressing HopU1 and exposed to the biotic stress may be painted with a solution of NBT as described in 33 (Cessna, S. G., V. E. Sears, M. B. Dickman, and P. S. Low. Oxalic acid, a pathogenicity factor for Sclerotinia sclerotiorum, suppresses the oxidative burst of the host plant. Plant Cell 12:2191-2200 (2000)). Likewise, DCF-DA is oxidized by ROS in cells and yields the green fluoroscent product DCF that can be viewed with fluorescence microscopy. (LeBel, C. P., H. Ischiropoulos, and S. C. Bondy. Evaluation of the probe 2′,7′-dichlorofluorescin as an indicator of reactive oxygen species formation and oxidative stress. Chem. Res. Toxicol. 5:227-231 (1992)). Plants expressing or over-expressing HopU1, for example, HopU1 transgenic plants, may produce reduced amounts of ROS, for example, significantly reduced amounts of ROS, as compared to a control plant. The control plant may be a plant that does not have the expression or over-expression of HopU1 or one that is not exposed to a biotic stress. The proper positive and negative controls will be known to one of skill in the art.

Optionally, plants expressing HopU1 polypeptides and/or those with under-expressed or decreased levels of one or more HopU1 substrates may be evaluated for the disease symptoms of the pathogen and their ability to grow in these plants using standard techniques known in the art. (Petnicki-Ocwieja, T., D. J. Schneider, V. C. Tam, S. T. Chancey, L. Shan, Y. Jamir, L. M. Schechter, M. D. Janes, C. R. Buell, X. Tang, A. Collmer, and J. R. Alfano. Genomewide identification of proteins secreted by the Hrp type III protein secretion system of Pseudomonas syringae pv. tomato DC3000. Proc. Natl. Acad. Sci. USA 99:7652-7657 (2002); and Wehling, M. D., M. Guo, Z. Q. Fu, and J. R. Alfano. The Pseudomonas syringae HopPtoV protein is secreted in culture and translocated into plant cells via the type III protein secretion system in a manner dependent on the ShcV type III chaperone. J. Bacteriol. 186:3621-3630 (2004)). For example, diseases symptoms and multiplication of the pathogen in planta may be determined at various time points as a measure of whether or not innate immunity is suppressed by over-expression of HopU1 in a plant and/or those plants with decreased levels of one or more HopU1 substrates.

HopU1 is less similar to other known Hops, sharing only 21% identity to HopO1-1 and HopO1-2; however, additional polypeptides having HopU1 activity may be identified according to the invention. Those skilled in the art will readily appreciate that the assays described earlier in this section can be adapted to screen for other polypeptides that have HopU1 activity that may be involved in suppressing the immune response, for example, to a biotic stress. This can be determined using a variety of methods and assays routine to one skilled in the art, for example, determining the ADP-ribosylation of a substrate of HopU1. Generally, a purified or partially purified candidate HopU1 polypeptide is incubated with a peptide comprising the ADP-ribosylation target sequence of HopU1 under conditions suitable for the HopU1 to ribosylate its target sequence of amino acids (i.e., protein, polypeptide). The particular conditions of the ADP-ribosylation may be determined empirically by one of skill in the art, or using the conditions that are described herein. The extent of ribosylation of the target substrate peptide is determined in the presence and absence of the candidate HopU1 polypeptide.

Known HopU1 polynucleotides can be used to identify additional HopU1 polynucleotides in a sample of interest, for example by Northern, Southern or Western blot analysis. Polypeptides are conveniently obtained by translation in an in vitro translation system, or by expression in a suitable host cell. To produce an expression vector, a polynucleotide encoding the desired polypeptide is operably linked to control elements for transcription and translation, and then transfected into a suitable host cell. Expression may be effected in prokaryotes such as E. coli, eukaryotic microorganisms such as the yeast Saccharomyces cerevisiae, or higher eukaryotes, such as plant, insect or mammalian cells. Following production, the protein is typically purified from the producing host cell by standard methods in protein chemistry in an appropriate combination, which may include ion exchange chromatography, affinity chromatography, and HPLC. Expression products are optionally produced with a sequence tag to facilitate affinity purification, which can subsequently be removed by proteolytic cleavage.

Such polypeptides can be tested, including but not limited to the characterization of suppression of the immune response, assays for the identification of HopU1 ADP-RT activity. Variants, fragments or domains of HopU1 polypeptides can be assessed for HopU1 activity and employed in the methods of the present invention.

In another example, HopU1 is labeled with a traceable substituent, such as biotin, a fluorescent group, an enzyme, a radioisotope, or a peptide group (e.g., FLG, HA, myc, or an immunoreactive peptide sequence), and then combined in a reaction solution with an isolated candidate binding partner, or with a mixture of components (such as a cell extract) in which compounds with HopU1 binding activity may be found. Formation of complexes with the labeled HopU1 is then detected (for example, by gel shift techniques or immunoprecipitation), and correlated with binding activity for HopU1.

Another approach is a co-expression system, using HopU1, a HopU1 fragment, or a candidate HopU1 polypeptide as bait. For example, a yeast two-hybrid screen system is employed, in which a HopU1 encoding sequence is fused to one part of the expression system, and a library of candidate binding partners is fused to the complementary component needed for expression of the marker. Cloned cells that express the activity of the marker contain an insert that comprises the encoding sequence for a HopU1 binding partner. Yeast two-hybrid screen systems are described generally in Bianchi et al., EMBO J., 16:1785, 1997. Reagents and suitable libraries are commercially available.

II. Method of Enhancing the Innate Immune Response of a Plant.

The invention is related to the discovery of HopU1 substrates and the use of these substrates for the enhancement of the innate immune response of a plant to a biotic stress. Exemplary HopU1 substrates are RNA-binding proteins (RBPs) that are glycine-rich RNA-biding proteins (GR-RBPs) and chloroplast RNA-biding proteins (CP-RBPs). The HopU1 substrates ADP-ribosylated by HopU1 were identified in Arabidopsis thaliana and include three CP-RBPs and two glycine-rich RNA-binding proteins (GR-RBPs). (See Table 1). To the inventor's knowledge, RNA binding proteins have not been described as a substrate for any ADP-RT, much less, HopU1.

In one aspect, the invention provides for a method of enhancing an innate immune system of a plant to a biotic stress. In one aspect, the method includes over-expressing a HopU1 substrate in a plant. In one aspect, the HopU1 substrate is a RNA binding protein, including but not limited to a glycine rich RNA binding protein or a chloroplast RNA binding protein. The HopU1 substrate is capable of being ADP-ribosylated by HopU1. It is also contemplated that certain residues of the HopU1 substrate may be altered to prevent ADP-ribosylation by HopU1, including those located within the RRM. Such residues include arginines and may be located within the RBP's RNA-recognition motif (RRM).

RNA binding proteins have a conserved RNA Recognition Motif (RRM). Chloroplast RNA binding proteins may have two RRMs and glycine rich RNA binding proteins may only have one RRM. The first RRM of cp29-like Chloroplast RBP (At2g377220.1) is from position 92 to position 165 and the second RRM is from position 205 to position 278. The first RRM of cp31-like Chloroplast RBP (At5g50250.1) is from position 114 to position 187 and the second RRM is from position 207 to position 291. The first RRM of AtRBP31 Chloroplast RBP (At4g2477.1) is from position 151 to position 224 and the second RRM would be from position 245 to 318. The RRM can be found from position 9 to position 82 in AtGRP7 (At2g21660.1) and position 7 to position 80 in AtGRP8 (At4g39260.1).

The HopU1 substrate when over-expressed in a plant will enhance the innate immune response of a plant that is subjected to a biotic stress. Examples of HopU1 substrates include but are not limited to cp29-like Chloroplast RBP (At2g377220.1) (accession numbers for protein and nucleotides are NP_{—}181259 and NM_{—}129278.3 respectively), cp31-like Chloroplast RBP (At5g50250.1) (accession numbers for protein and nucleotide are NP_{—}199836 and NM_{—}124404.3, respectively), At RBP31 Chloroplast RBP (At4g2477.1) (accession numbers for protein and nucleotide are NP_{—}194208 and NM_{—}118610.3, respectively), Arabidopsis glycine rich RNA binding proteins AtGRP7 (accession numbers for the protein and nucleotide are NP_{—}179760 and NM_{—}127738, respectively), glycine rich RNA binding protein At GRP8 (At4g39260.1) (accession numbers for protein and nucleotide are NP_{—}195637 and NM_{—}120087.3, respectively) and variants thereof, including conserved amino acid substitutions.

As described above, a polynucleotide of a HopU1 substrate may be introduced into either a prokaryotic or eukaryotic plant or animal host cell through the use of a vector, plasmid or construct. An expression cassette containing a promoter operably linked to a nucleic acid that encodes the HopU1 substrate may be introduced into suitable host cells, including for example, bacterial, insect, fungal, plant or animal cells. Either constitutive, induced, or regulated promoters can be used in the present invention. Promoters suitable for use in eukaryotic host cells are well known to those of skill in the art and are described elsewhere in the specification.

Additional HopU1 substrates may be isolated or identified from any suitable source, for example, from genomic or cDNA libraries, plant extracts, or plant or animal cells. Extracts may be from any plant, dicot or monocot, such as rice, wheat, corn, tomato, corn, soybean, sunflower, sorghum, canola, alfalfa, cotton, barley or millet. Extracts may be from any plant, insect, yeast, or mammalian cells. Examples of mammalian cells also include those cells that grow in cell culture, such as HeLa, 22Rv1 (human prostatic cells), and 293T cells and the like. In one aspect, HopU1 substrates may be identified either in vivo or in vitro using any number of methods.

Functional assays for ADP-ribosyltransferase can be conducted by incubating the HopU1 polypeptide with a candidate HopU1 substrate or an extract suspected of containing a HopU1 substrate (see Example 7)), in the presence of NAD⁺ or an analog labeled with a radioisotope such as ³² P, biotin, or a fluorescent group. (Sun, J., and J. T. Barbieri. Pseudomonas aeruginosa ExoT ADP-ribosylates CT10 regulator of kinase (Crk) proteins. J. Biol. Chem. 278:32794-32800 (2003)). Substrates that contain the biotinylated NAD+ may be isolated and concentrated with avidin affinity chromatography and identified. (Zhang, J. Use of biotinylated NAD to label and purify ADP-ribosylated proteins. Methods Enzymol. 280:255-265 (1997); and Zhang, J., and S. H. Snyder. Purification of a nitric oxide-stimulated ADP-ribosylated protein using biotinylated beta-nicotinamide adenine dinucleotide. Biochem. 32:2228-2233 (1993)).

ADP-ribosylation can be monitored by incorporation of the label into the protein phase, by a change of size of the target protein (measurable, for example on a protein gel), or by detection of ADP ribose polymers on the target (for example, using commercially available antibody specific for ADP-ribose polymers, or by physical-chemical mechanisms, such as mass spectrometry). Known ADP-RT inhibitors like novobiocin can be used to verify the specificity of the assay. Polypeptides with confirmed ADP-RT ribosylation can then be used in methods of the present invention, for example, enhancing the innate immune system of a plant or further characterized. For example, two-dimensional PAGE may be performed on ADP-RT reactions and detected for example by autoradiology. Radiolabled spots may be isolated from the gel and digested with enzymes such as endopeptidases and analyzed with tandem mass spectrometry to identify the HopU1 substrate. The cDNAs of the identified HopU1 substrates may be isolated or amplified from the plant or animal nucleic acids, a nucleic acid library, or produced by synthetic chemistry. The polynucleotide sequence of the HopU1 substrate may be inserted into any of the many available expression vectors and cell systems using reagents that are well known in the art. For example, HopU1 substrate sequences may be ligated into a GST fusion vector, transformed in bacteria and purified with glutathione Sepharose and subjected to in vitro ADP-RT assays with HopU1, for example, HopU1 His as described in Example 3. Two-dimensional PAGE and autoradiology be performed on the reactions to determine whether the candidate HopU1 substrates were ADP-ribosylated by HopU1 in vitro.

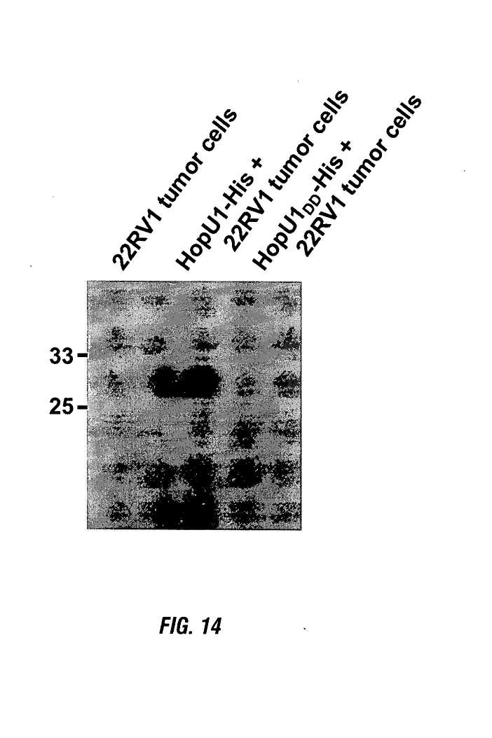

In one aspect, mammalian substrates of HopU1 may be identified using any of methods described herein or known to one skilled in the art. For example, extracts from mammalian cells may be added to an ADP-RT reaction mix containing HopU1, for example, HopU1His or HopU1_{DD}-His and then the samples separated, using for example, a SDS-PAGE. Ribosylation may be detected as described herein, including exposing ADP-RT reactions separated by SDS-PAGE to film where protein bands may be detected and further analyzed. (See FIG. 14 which shows a protein band of about 29 kDa that was ADP-ribosylated in a HopU1-dependent manner. The molecular mass of this ADP-ribosylated protein is similar to several of the substrates that were ADP-ribosylated in plant extracts by HopU1. Mammalian substrates of HopU1 may be identified in vitro and in vivo experiments, using for example, heterologous delivery of HopU1 into animal cells via the P. aeruginosa TTSS. In another aspect, the method includes localizing HopU1 and its substrates to determine if they co-localize. The Barbieri protocol may be used for preparing the cell line extracts, which includes an acetone precipitation step. 1. Sun, J., and J. T. Barbieri. 2003. Pseudomonas aeruginosa ExoT ADP-ribosylates CT10 regulator of kinase (Crk) proteins. J. Biol. Chem. 278:32794-32800. [³²P]-NAD ART (ADP-ribosyltransferase) assays may be performed as described herein above and subjected to 2D SDS-PAGE and autoradiography. Protein spots that align with ADP-RT activity spots may be identified using MALDI-TOF and Q-TOF MS. Mammalian HopU1 substrates include but are not limited to RNA binding proteins.

In another aspect, the HopU1 substrates are substrates of HopU1 identified in planta. The ADP-ribosylation of substrates by HopU1 can be determined using transient expression in a plant, for example, in Nicotiana benthamiana using binary vectors that express a HopU1 polypeptide and a HopU1 substrate or a candidate HopU1 substrate. The HopU1 polypeptide and HopU1 substrate or a candidate HopU1 substrate may contain epitopes for detection by antibodies as necessary, for example, HA, FLAG, HopU1, GRP-RBP or CP-RBP antibodies. Extracts from the transiently expressed plant may be analyzed by using a combination of SDS-PAGE gels and immunoblots using antibodies that recognize the HopU1 polypeptide and HopU1 substrate or a candidate HopU1 substrate, for example, HA, FLAG, HopU1, GRP-RBP or CP-RBP antibodies. If a substrate is ADP-ribosylated by HopU1, the substrate's molecular mass would be expected to increase by 541 Da increments (the molecular mass of ADP-ribose) as indicated by a shift in mobility.

In another aspect, the HopU1 substrates are substrates of HopU1 identified in vivo. In one aspect, ³²P labeled NAD+ is introduced into plant suspension or mammalian cultured cells that express HopU1 and then extracts from these cells are analyzed with SDS-PAGE and autoradiography. (Sun, J., and J. T. Barbieri. Pseudomonas aeruginosa ExoT ADP-ribosylates CT10 regulator of kinase (Crk) proteins. J. Biol. Chem. 278:32794-32800 (2003)). Protein spots that co-migrate with the ³²P labeled proteins may be identified using mass spectrometry.

In one aspect, a polypeptide, variant or fragment of the HopU1 substrate may be over-expressed in a plant. The HopU1 substrate that is to be over-expressed may be mutated in its amino acid residues that are suspected or known to be ribosylated by HopU1 so that the HopU1 substrate is not able to be modified by HopU1. For example, the number of amino acid residues that are ADP-ribosylated may be determined using mass spectrometry. HopU1 substrate polynucleotides or candidate HopU1 substrate polynucleotides may be cloned into a vector that fuses the substrate to an affinity tag so that it can be partially purified, subjected to ADP-RT reactions, SDS-PAGE, and liquid chromatography coupled with tandem mass spectrometry to determine the mass difference between a modified or unmodified substrate. The peptides may be analyzed with ESI-MS-MS to identify the residues that are ADP-ribosylated. The amino acid or amino acids that is/are modified within the substrate may be altered with site-directed mutagenesis, purified with affinity chromatography and retested in ADP-RT assays to confirm that the modified HopU1 substrate is no longer ADP-ribosylated by HopU1. The modified HopU1 substrate lacking residues that are ADP-ribosylated by HopU1 may be introduced into a plant either transiently or transgenically and over-expressed into a plant to enhance the innate immune response.

Enhancement of the plant's innate immune response may be achieved through over-expression of one or more HopU1 substrates. As discussed above, one of skill will recognize the appropriate expression vector and promoter to use to express the HopU1 substrate. Exemplary promoters for this embodiment include constitutive promoters or promoters that are preferentially active in all or most plant tissues including, for example, those active in tissues of leaves, stems and roots.

Leaf-preferred and stem-preferred promoters are known in the art. See, for example, Yamamoto et al. (1997) Plant J. 12(2):255-265; Kwon et al. (1995) Plant Physiol. 105:357-67; Yamamoto et al. (1995) Plant Cell Physiol. 35(5):773-778; Gotor et al. (1993) Plant J. 3:509-18; Orozco et al. (1993) Plant Mol. Biol. 23(6):1129-1138; Baszczynski et al. (1988) Nucl. Acid Res. 16:5732; Mitra et al. (1995) Plant Molecular Biology 26:35-93; Kayaya et al. (1995) Molecular and General Genetics 258:668-675; and Matsuoka et al. (1993) Proc. Natl. Acad. Sci. USA 90(20):9586-9590. Senescence regulated promoters are also of use, such as, SAM22 (Crowell et al. (1992) Plant Mol. Biol. 18:559-566). See also U.S. Pat. No. 5,589,052, herein incorporated by reference.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 15(3):533-553 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean).

Exemplary transfection or transformation techniques to achieve over-expression are described elsewhere in the specification. Modulation of HopU1 substrate expression in a plant or plant cell provides a novel strategy for enhancing the innate immune system of a plant exposed to a biotic stress. The evaluation of the effect of HopU1 substrate over-expression on the plant's innate immune response to a biotic stress may also include evaluation of phenotypic changes. As described in Example 2, plants may be treated with bacterial flagellin flg22 as a biotic stress to trigger the innate immune response. Examples of phenotypic changes to a biotic stress include but are not limited to maintained or increased callose deposition, elicitation of the HR, oxidative burst (ROS levels), phenolic accumulation, and decreased pathogen multiplication or disease symptoms of the pathogen. In one aspect, the phenotypic changes are measured as compared to a control that does not have over-expression of the HopU1 substrate. In one aspect, the phenotypic changes are measured as compared to a control that over-expresses both HopU1 and the HopU1 substrate.

### EXAMPLES

### Example 1

### hop U1 Encodes a Putative ADP-RT

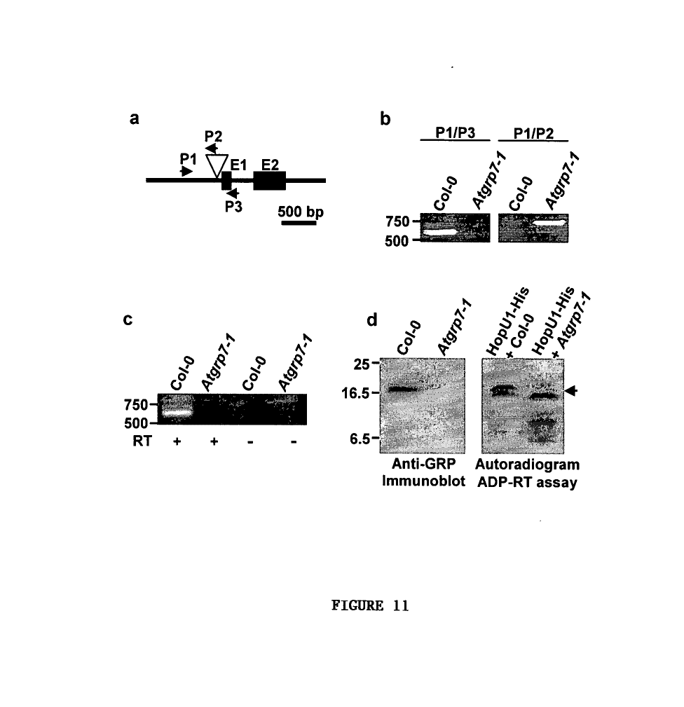

To begin to characterize the DC3000 effector genes that potentially encode ADP-RTs we focused on hopU1, which is downstream of an apparent type III promoter and the shcF type III chaperone gene and the hopF2 effector gene in the DC3000 chromosome (FIG. 1b). Semi-quantitative RT-PCR experiments indicated the hopU1 gene is transcribed and its expression was elevated when DC3000 was grown in a medium that induces the expression of the T3SS (FIG. 1c). Three regions of HopU1 share similarity with known ADP-RTs (FIG. 1a). HopU1 was type III-injected into plant cells based on adenylate cyclase translocation assays (FIG. 11a). A DC3000 ΔhopU1 mutant was reduced 6 fold in its ability to multiply in plant tissue and cause disease symptoms in A. thaliana Col-0 (FIG. 11b).

### Example 2

### HopU1 Suppresses Plant Innate Immunity

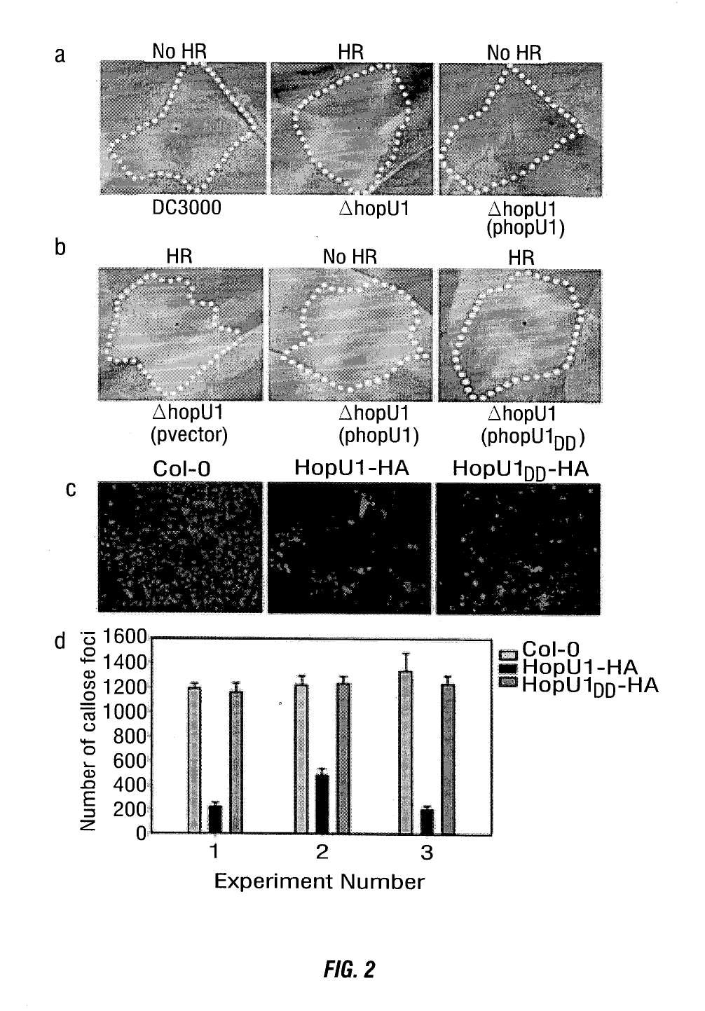

We earlier reported that DC3000 mutants defective in type III effectors that can suppress the hypersensitive response (HR), a programmed cell death of plant cells associated with innate immunity, often display an enhanced ability to elicit an HR (Jamir, Y. et al. Identification of Pseudomonas syringae type III effectors that suppress programmed cell death in plants and yeast. Plant J. 37:554-565 (2004)). To investigate whether the ΔhopU1 mutant shared this phenotype we infiltrated wild type DC3000 and the ΔhopU1 mutant at different cell densities into Nicotiana tabacum cv. Xanthi (tobacco). We consistently found that the ΔhopU1 mutant elicited an HR in tobacco at cell densities below the threshold needed for wild type DC3000 (FIG. 2a). When hopU1 was expressed in trans in the ΔhopU1 mutant it complemented this phenotype (FIG. 2a). We also assessed cell death by measuring the amount of ion leakage from plant cells and found that the ΔhopU1 mutant caused more cell death than DC3000 and expression of hopU1 in trans reduced the amount of cell death to wild type levels (FIG. 11a). Taken together, these results provide genetic evidence that HopU1 acts as a suppressor of the nonhost HR.

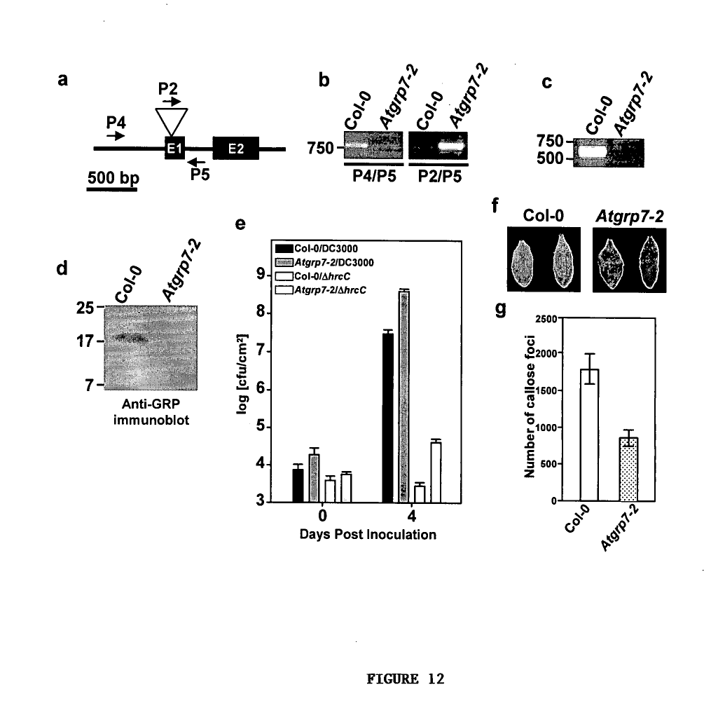

To determine whether the predicted ADP-RT activity of HopU1 was required for suppression of the HR, we ectopically expressed in the ΔhopU1 mutant a HopU1 derivative (HOPU1_{DD}) that had its glutamic acid residues in its putative ADP-RT active site (FIG. 1a) substituted with aspartic acids. This strain elicited an enhanced HR similar to the ΔhopU1 mutant control suggesting that the suppression of the HR required a functional ADP-RT active site (FIG. 2b). Ion leakage conductivity assays indicated that the ΔhopU1 mutant expressing HopU1_{DD} caused similar amounts of cell death as the ΔhopU1 mutant control (FIG. 12b). Thus, the HR suppression activity of HopU1 requires a functional ADP-RT catalytic site.

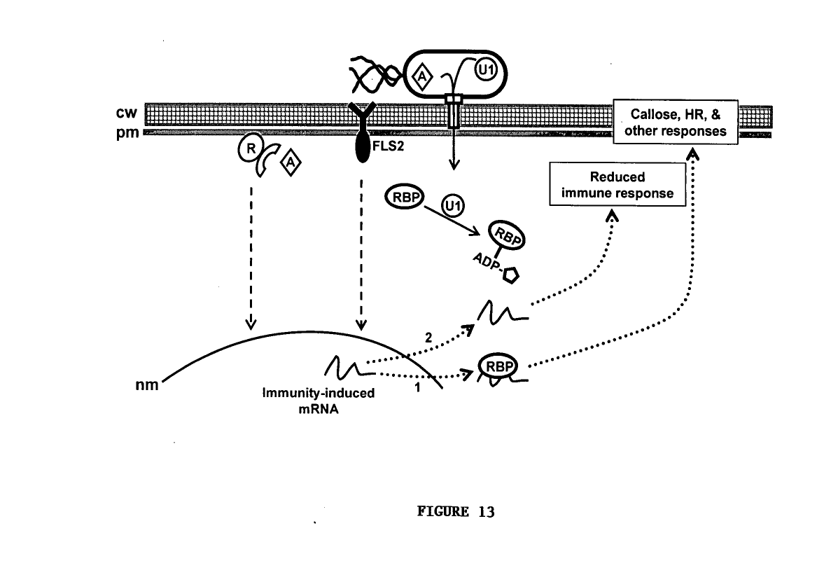

We reasoned that HopU1 may be capable of suppressing other innate immune responses. To test this we generated transgenic A. thaliana Col-0 plants that constitutively produced HopU1 fused to a hemagglutinin epitope (HopU1-HA). Bacterial flagellin often acts as a pathogen-associated molecular pattern (PAMP), a conserved molecule from a microorganism recognized by animal and plant innate immune systems (Numberger, T., Brunner, F., Kemmerling, B. & Piater, L. Innate immunity in plants and: striking similarities and obvious differences. Immunol. Rev. 198:249-266 (2004); Akira, S., Uematsu, S. & Takeuchi, O. Pathogen recognition and innate immunity. Cell 124:783-801 (2006); and Jones, J. D. & Dangl, J. L. The plant immune system. Nature 444:323-329 (2006)). A conserved peptide from bacterial flagellin, flg22, has been shown to be effective at triggering callose (β-1-3 glucan) deposition (Gomez-Gomez, L., Felix, G. & Boller, T. A single locus determines sensitivity to bacterial flagellin in Arabidopsis thaliana. Plant J. 18:277-284 (1999)). The HopU1-HA-expressing transgenic plants treated with flg22 produced significantly reduced amounts of callose compared to wild type plants (FIGS. 2c and d). These plants also elicited a delayed atypical HR in response to the type III effector AvrRpt2, which is recognized by the RPS2 resistance protein present in A. thaliana Col-0 (Whalen, M. C., Innes, R. W., Bent, A. F. & Staskawicz, B. J. Identification of Pseudomonas syringae pathogens of Arabidopsis and a bacterial locus determining avirulence on both Arabidopsis and soybean. Plant Cell 3:49-59 (1991)) (FIG. 13). Together these data indicate that HopU1 can suppress innate immune responses that are triggered by both a type III effector protein or a PAMP.

### Example 3

### HopU1 is an Active ADP-RT

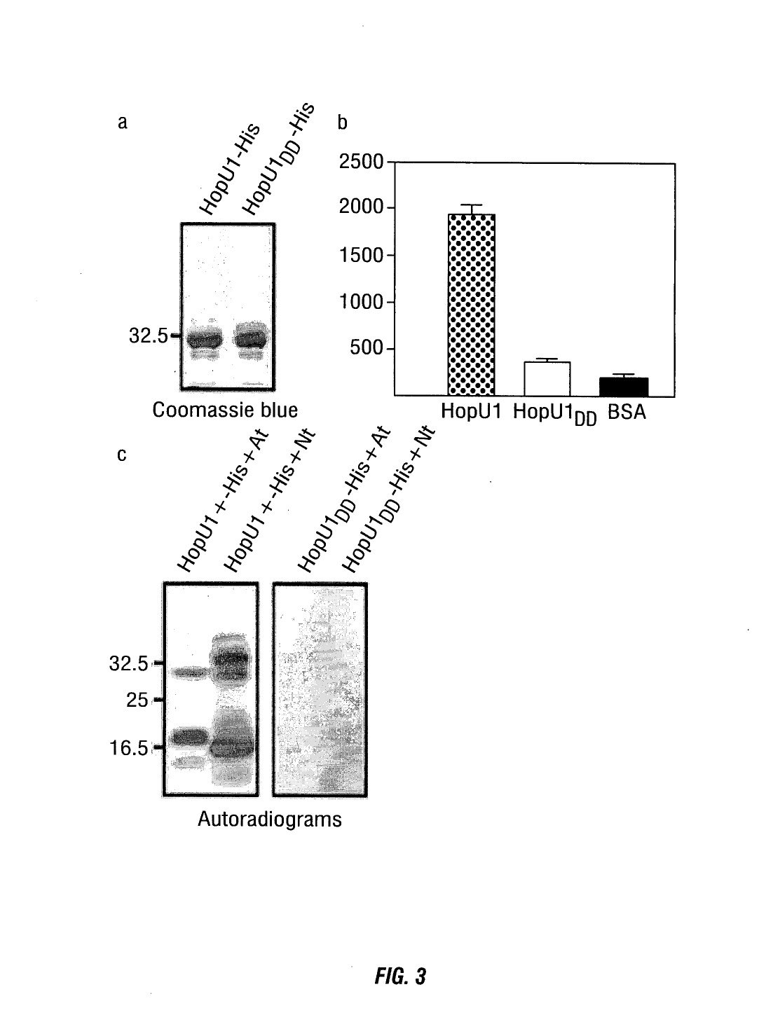

To explore whether HopU1 indeed possessed ADP-RT activity we purified recombinant HopU1 and the catalytic site mutant (HOPU1_{DD}), both fused to histidine affinity tags (FIG. 3a). The activity of recombinant HopU1-His was tested with poly-L-arginine, an artificial substrate for many ADP-RTs that can modify arginine residues. HopU1-His was capable of ADP-ribosylating poly-arginine in the presence of [³²P]-NAD, while the HopU1_{DD}-His mutant incorporated radioactivity in amounts similar to the BSA control (FIG. 3b). Therefore, HopU1 is an active ADP-RT that can modify arginine residues.

We next examined whether HopU1-His was capable of using plant proteins as substrates. Crude protein extracts from the leaves of A. thaliana ecotype Col-0 and tobacco were used in ADP-RT reactions. ADP-RT reactions were separated by SDS-PAGE, and subjected to autoradiography (FIG. 3c). At least two proteins in A. thaliana extracts and three in tobacco were ADP-ribosylated by HopU1-His. No labelled products were detected from reactions using the inactive HopU1_{DD}-His (FIG. 3c). Therefore, HopU1-His can use several Arabidopsis and tobacco proteins as substrates.

### Example 4

### RNA-Binding Proteins are Substrates

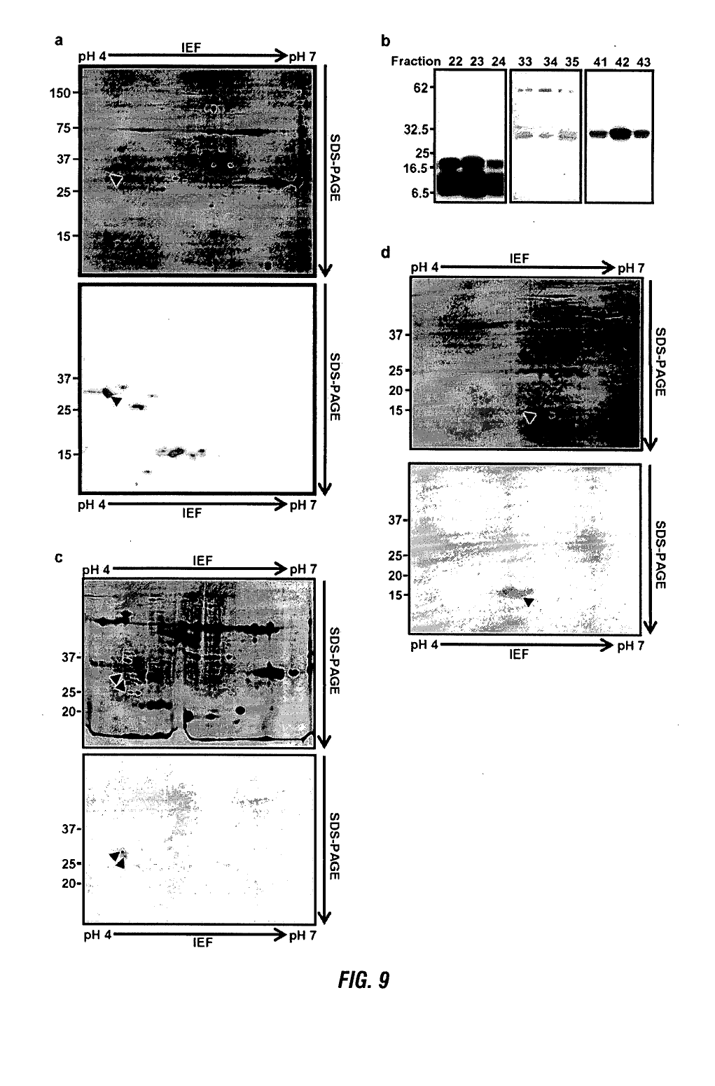

To determine the identity of the proteins ADP-ribosylated by HopU1 in A. thaliana extracts we separated ADP-RT reactions with two-dimensional (2D) PAGE followed by autoradiography (for representative examples, see FIG. 14). One well separated protein spot stained with Coomassie blue has the identical migration as a radiolabelled ADP-RT activity spot. It was analyzed with tandem mass spectrometry and corresponded to chloroplast RNA-binding protein (CP-RBP) AtRBP31 (Ohta, M., Sugita, M. & Sugiura, M. Three types of nuclear genes encoding chloroplast RNA-binding proteins (cp29, cp31 and cp33) are present in Arabidopsis thaliana: presence of cp31 in chloroplasts and its homologue in nuclei/cytoplasms. Plant Mol. Biol. 27:529-539 (1995)) (Table 1). The other ADP-RT activity spots did not co-migrate with visible Coomassie blue-stained protein spots suggesting that these proteins were in lower abundance. To enrich for less abundant substrates more concentrated A. thaliana extracts were made and fractionated using ion exchange chromatography. ADP-RT assays were performed on aliquots of each fraction (FIG. 9b). Coomassie blue-stained protein spots that had identical migration patterns as ADP-RT modified protein spots visible on autoradiograms were analyzed with tandem mass spectrometry. In total, we found three CP-RBPs and two glycine-rich RNA-binding proteins (GR-RBPs), AtGRP7 and AtGRP8²⁸, to be ADP-ribosylated by HopU1-His (Table 1).

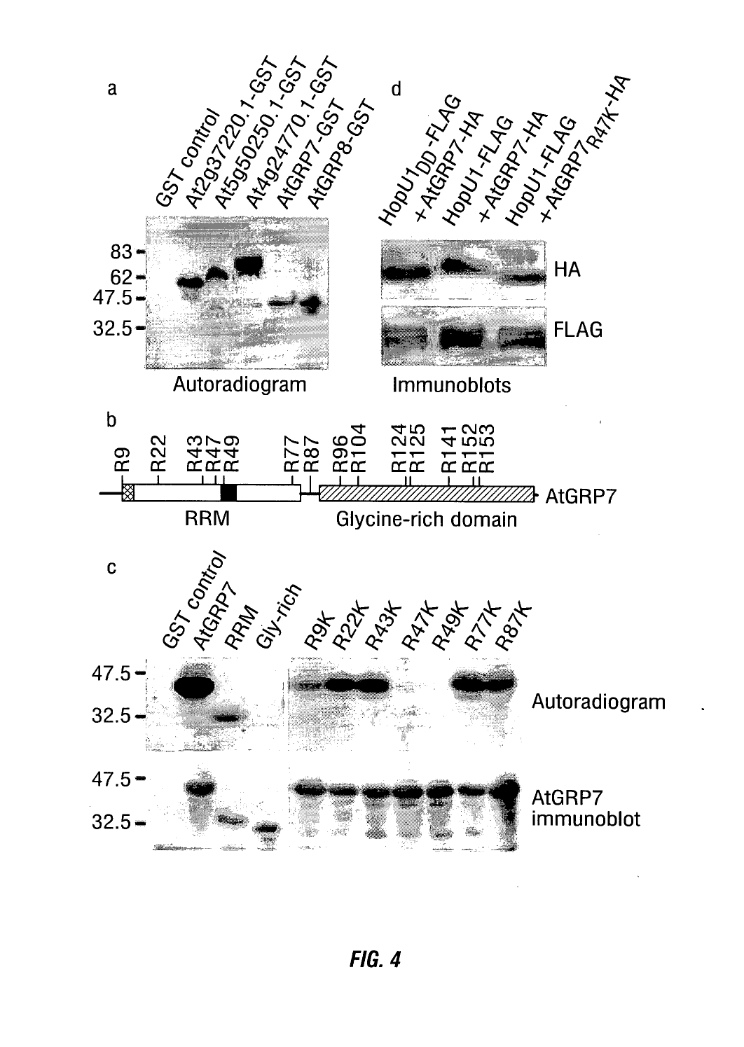

To confirm that the identified CP-RBPs and GR-RBPs were substrates for HopU1-His, we constructed glutathione S-transferase (GST) fusions. Substrate-GST fusions were partially purified with glutathione Sepharose or used as crude lysates in ADP-RT reactions. Each of the substrate-GST fusions could act as substrates for HopU1-His (FIG. 4a). Other plant protein-GST fusion corresponding to proteins neighbouring HopU1 substrates on 2D polyacrylamide gels were not ADP-ribosylated by HopU1-His. Thus, these CP-RBPs and GR-RBPs indeed are in vitro substrates for the HopU1 ADP-RT. We did not find additional HopU1 substrates in our extensive mass spectrometry analyses suggesting that the in vivo targets of HopU1 are among these proteins.

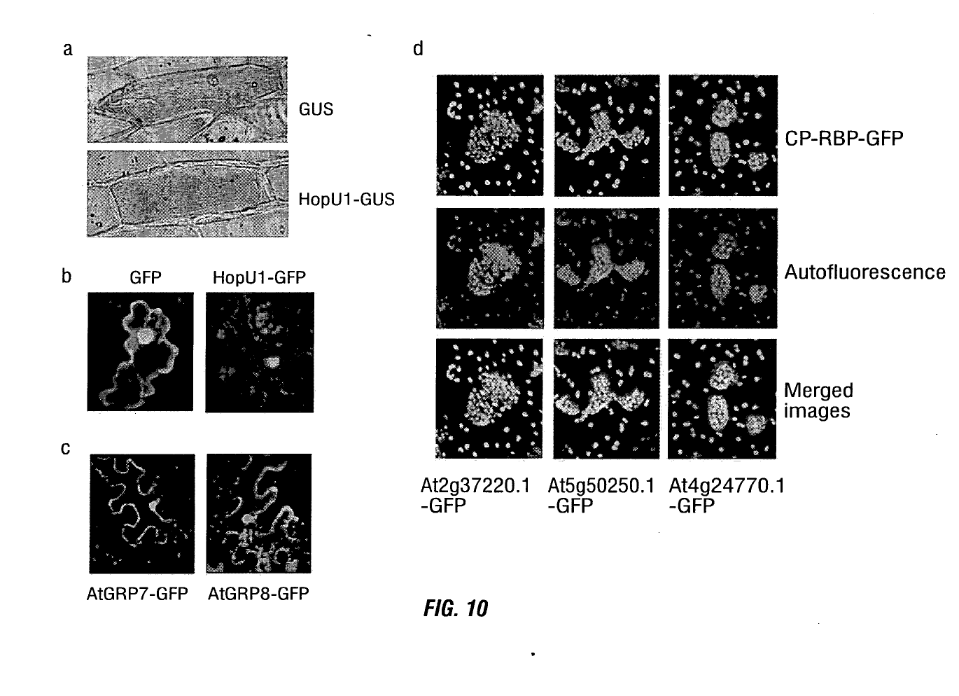

CP-RBPs and GR-RBPs belong to a group of RNA-binding proteins that all have in common an RNA-recognition motif (RRM), a protein domain demonstrated to be necessary and sufficient for RNA binding (Burd, C. G. & Dreyfuss, G. Conserved structures and diversity of functions of RNA-binding proteins. Science 265:615-621 (1994); and Jessen, T. H., Oubridge, C., Teo, C.-H., Pritchard, C. & Nagai, K. Identification of molecular contacts between the U1 A small nuclear ribonucleoprotein and U1 RNA. EMBO 10:3447-3456 (1991)). Localization experiments found that HopU1-GFP (and HopU1-GUS) and the GR-RBPs AtGRP7 and AtGRP8 GFP fusion proteins were similarly localized to the cytoplasm and possibly to the nucleus, while the CP-RBP-GFP fusions were discretely localized to the chloroplast (FIG. 10). Because of our localization experiments and the association of GR-RBPs with abiotic and biotic stress (Gomez, J. et al. A gene induced by the plant hormone abscisic acid in response to water stress encodes a glycine-rich protein. Nature 334:262-264 (1988); and Naqvi, S. M. et al. A glycine-rich RNA-binding protein gene is differentially expressed during acute hypersensitive response following Tobacco Mosaic Virus infection in tobacco. Plant Mol. Biol. 37:571-576 (1998)), we focused on the GR-RBPs as putative physiological targets of HopU1.

The GR-RBP AtGRP7 and AtGRP8 are homologous to each other sharing 76.9% identity and likely perform related functions. AtGRP7 has been shown to bind RNA and influence mRNA oscillations in response to circadian rhythms at the post-transcriptional level (Heintzen, C., Nater, M., Apel, K. & Staiger, D. AtGRP7, a nuclear RNA-binding protein as a component of a circadian-regulated negative feedback loop in Arabidopsis thaliana. Proc. Natl. Acad. Sci. USA 94:8515-8520 (1997); and Staiger, D., Zecca, L., Wieczorek Kirk, D. A., Apel, K. & Eckstein, L. The circadian clock regulated RNA-binding protein AtGRP7 autoregulates its expression by influencing alternative splicing of its own pre-mRNA. Plant J. 33:361-371 (2003)). AtGRP7 contains 14 arginine residues that represent putative sites of ADP-ribosylation (FIG. 4b). We found that the RRM domain-GST fusion was ADP-ribosylated by HopU1-His, while the glycine-rich domain-GST fusion was not (FIG. 4c). To determine the arginine residues required for ADP-ribosylation, each arginine of RRM was individually mutated to lysine in full length AtGRP7-GST fusions. When arginines in positions 47 or 49 of AtGRP7 were substituted with lysine these AtGRP7-GST derivatives were no longer ADP-ribosylated suggesting that one of these residues is the site of the ADP-ribose modification while the other may be required for substrate recognition (FIG. 4c). Interestingly, based on the RRM domain structure in other RNA-binding proteins both of these residues would likely be solvent-exposed (Maris, C., Dominguez, C. & Allain, F. H. The RNA recognition motif, a plastic RNA-binding platform to regulate post-transcriptional gene expression. FEBS J. 272:2118-2131 (2005)). Moreover, the arginine in position 49 is within RNP1, the most conserved region of the RRM domain and one that has been directly implicated in RNA-binding Burd, C. G. & Dreyfuss, G. Conserved structures and diversity of functions of RNA-binding proteins. Science 265, 615-621 (1994)).

To determine if AtGRP7 can be ADP-ribosylated by HopU1 in planta, we co-expressed HopU1-FLAG or HopU1_{DD}-FLAG and AtGRP7-HA or AtGRP7R_{47K}-HA (an AtGRP7 derivative that cannot be ADP-ribosylated) in N. benthamiana using Agrobacterium transient assays. After 40 h, plant extracts isolated from leaf tissue were separated on SDS-PAGE gels and analyzed with immunoblots using anti-FLAG or anti-HA antibodies. We consistently observed an increase in the molecular mass of AtGRP7-HA when it was expressed in planta with HopU1-FLAG, but not when expressed with HopU1_{DD}-FLAG or when HopU1-FLAG was expressed with AtGRP7R_{47K}-HA (FIG. 4d) suggesting that the increased molecular mass was due to ADP-ribosylation. Therefore, HopU1-FLAG can ADP-ribosylate AtGRP7-HA inside the plant cell.

### Example 5

### A Atgrp7 Mutant is More Susceptible

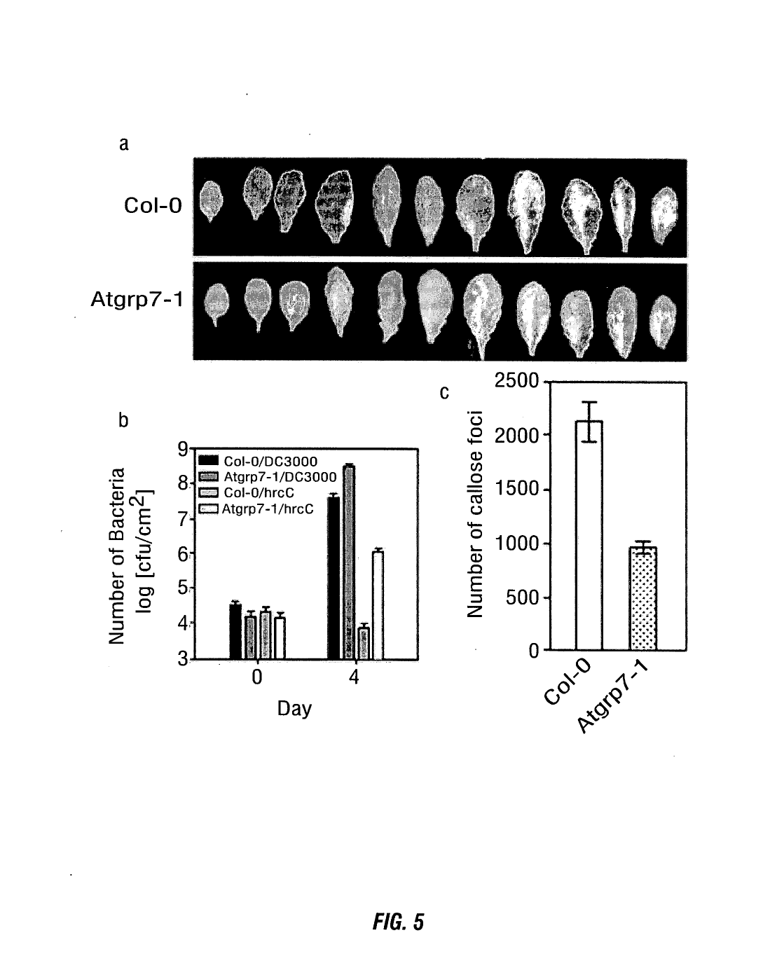

To determine the involvement of AtGRP7 in plant innate immunity we identified an A. thaliana SALK homozygous T-DNA insertion line (Alonso, J. M. et al. Genome-wide insertional mutagenesis of Arabidopsis thaliana. Science 301, 653-657 (2003)) in the AtGRP7 locus, and confirmed that it did not produce AtGRP7 mRNA and protein (FIG. 11). To determine if this mutant, designated Atgrp7-1, was altered in its responses to P. syringae, we infected wild type A. thaliana Col-0 and the Atgrp7-1 mutant plants with DC3000 and a ΔhrcC mutant defective in the T3SS. DC3000 caused enhanced disease symptoms on Atgrp7-1 mutant plants compared to wild type Col-0 (FIG. 5a). Each strain grew to higher levels in Atgrp7-1 plants compared to wild type Col-0 plants (FIG. 5b), indicating that Atgrp7-1 plants were more susceptible to P. syringae. The growth difference was even more pronounced for the ΔhrcC mutant, which is likely due to the fact that this strain cannot inject any type III effectors, many of which suppress innate immunity. Importantly, we found that flg22-induced callose deposition was reduced in Atgrp7-1 plants compared to wild type A. thaliana Col-0 (FIG. 5c), further supporting that Atgrp7-1 mutant plants were impaired in their innate immune responses. Similar phenotypes were observed for an independent T-DNA mutant designated Atgrp7-2 (FIG. 12).

### Example 6

### Discussion

Several plant pathogen type III effectors are known to suppress outputs of innate immunity (Abramovitch, R. B., Anderson, J. C. & Martin, G. B. Bacterial elicitation and evasion of plant innate immunity. Nat. Rev. Mol. Cell. Biol. 7:601-611 (2006)). However, the enzymatic activity and substrates for these type III effectors remain poorly understood (Grant, S. R., Fisher, E. J., Chang, J. H., Mole, B. M. & Dangl, J. L. Subterfuge and Manipulation: Type III Effector Proteins of Phytopathogenic Bacteria. Annu Rev Microbiol 60:425-449 (2006)). Indeed, there are only two cases that both an enzymatic activity and a substrate for a plant pathogen type III effector are known (Axtell, M. J. & Staskawicz, B. J. Initiation of RPS2-specified disease resistance in Arabidopsis is coupled to the AvrRpt2-directed elimination of RIN4. Cell 112:369-377 (2003); Mackey, D., Belkhadir, Y., Alonso, J. M., Ecker, J. R. & Dangl, J. L. Arabidopsis RIN4 is a target of the type III virulence effector AvrRpt2 and modulates RPS2-mediated resistance. Cell 112:379-389 (2003); and Shao, F. et al. Cleavage of Arabidopsis PBS1 by a bacterial type III effector. Science 301:1230-1233 (2003)). The substrates of the HopU1 ADP-RT suggest a novel strategy utilized by bacterial pathogens to modulate plant innate immunity by indirectly affecting host RNA status. That is, GR-RBPs may act as key post-transcriptional regulators through either the trafficking, stabilization, or processing of specific mRNAs in response to pathogen stress and the ADP-ribosylation of the GR-RBPs by HopU1 may disrupt their activity (FIG. 13). By disabling the function of GR-RBPs the pathogen may reduce the amount of immunity-related mRNAs available in the plant and tip the balance of the interaction in favor of the pathogen.

To our knowledge, this is the first demonstration of an active ADP-RT virulence protein in a plant pathogen. The DC3000 genome encodes at least two additional putative ADP-RTs, HopO1-1 and HopO1-2. These ADP-RTs possess low similarity with HopU1 and, therefore, likely target other plant proteins. ADP-RTs are well characterized in animal pathogens. However, the ADP-RT substrates described here are novel. Interestingly, IpaH9.8, a type III effector from the animal pathogen Shigella flexneri was recently shown to bind the RRM-containing mammalian splicing factor U2AF (Maris, C., Dominguez, C. & Allain, F. H. The RNA recognition motif, a plastic RNA-binding platform to regulate post-transcriptional gene expression. FEBS J. 272:2118-2131 (2005)) resulting in the suppression of pro-inflammatory cytokines (Okuda, J. et al. Shigella effector IpaH9.8 binds to a splicing factor U2AF(35) to modulate host immune responses. Biochem. Biophys. Res. Commun. 333:531-539 (2005)). The enzymatic activity of IpaH9.8 is presently unknown. The protein targets of HopU1 and IpaH9.8 suggest that animal and plant pathogens may alter RNA metabolism to quell the eukaryotic immune response.

### Example 7

### Methods Summary

Bacterial strains (and plasmids) and primers used in this work are listed in Tables 2 and 3, respectively. A ΔhopU1 mutant was made by homologous recombination. Expression of hopU1 was assessed with RT-PCR (Guo, M. et al. Pseudomonas syringae type III chaperones ShcO1, ShcS1, and ShcS2 facilitate translocation of their cognate effectors and can substitute for each other in the secretion of HopO1-1. J. Bacteriol. 187:4257-4269 (2005)). Construction of A. thaliana Col-0 transgenic plants was done using the Agrobacterium-mediated floral dip method (Bechtold, N., Ellis, J. & Pelletier, G. In planta Agrobacterium mediated gene transfer by infiltration of adult Arabidopsis thaliana plants. C. R. Acad. Sci. Ser. III Sci. Paris 316:1194-1199 (1993)). DC3000 strains were tested for their ability to elicit an HR on Nicotiana tabacum cv. Xanthi by infiltrating strains resuspended in 5 mM MES (pH 5.6) at a cell density of 1×10⁶ or 1×10⁷ cells/ml. Pathogenicity assays with A. thaliana plants were performed by spray-inoculation with bacterial suspensions (Espinosa, A., Guo, M., Tam, V. C., Fu, Z. Q. & Alfano, J. R. The Pseudomonas syringae type III-secreted protein HopPtoD2 possesses protein tyrosine phosphatase activity and suppresses programmed cell death in plants. Mol. Microbiol. 49:377-387 (2003)). Seeds of the Arabidopsis SALK_{—}039556.21.25.x and SALK_{—}051743.42.85.x lines, corresponding to the Atgrp7-1 and Atgrp7-2 mutants, respectively, were obtained from the Arabidopsis Biological Resource Center at Ohio State University. A. thaliana plants were infiltrated with 1 μM of flg22 and stained with aniline blue for callose as described (Gomez-Gomez, L., Felix, G. & Boller, T. A single locus determines sensitivity to bacterial flagellin in Arabidopsis thaliana. Plant J. 18:277-284 (1999)). Leaves were examined with a Zeiss AxioPlan2 Imaging System microscope with fluorescence. The poly-L-arginine assay was done as previously described (Coye, L. H. & Collins, C. M. Identification of SpyA, a novel ADP-ribosyltransferase of Streptococcus pyogenes. Mol. Microbiol. 54:89-98 (2004)). The ADP-RT assay was adapted from a described protocol (Sun, J. & Barbieri, J. T. Pseudomonas aeruginosa ExoT ADP-ribosylates CT10 regulator of kinase (Crk) proteins. J. Biol. Chem. 278:32794-32800 (2003)0. AtGRP7-GST site-directed mutant constructs were made using the QuikChange Site-Directed Mutagenesis kit (Stratagene). In planta HopU1 ADP-RT assays with AtGRP7 were done using Agrobacterium delivery as described (Jamir, Y. et al. Identification of Pseudomonas syringae type III effectors that suppress programmed cell death in plants and yeast. Plant J. 37:554-565 (2004)) and assessed with immunoblots.

Methods

Bacterial Strains and Nucleotide Primers.

Bacterial strains and plasmids used in this work are listed in Table 2. The primers used are listed in Table 3.

Construction of the DC3000 ΔhopU1 Mutant.

A DC3000 ΔhopU1 mutant was made by amplifying a 2 kb region upstream and downstream of hopU1 using PCR with primer sets P1058/P1059 and P1078/P1079. The DNA fragment upstream of hopU1 was ligated into pHP45Ω using BamHI and HindIII sites. The DNA fragment downstream of hopU1 was ligated into the pHP45Ω derivative containing the upstream fragment using XbaI and SacI restriction enzymes such that the hopU1 flanking regions were on either side of an omega fragment in the same orientation. This cassette was ligated into the broad host range vector pRK415 using BamHI and SacI restriction enzymes. The resulting construct was electroporated into DC3000 and homologous recombination was selected for by selecting for retention of the antibiotic marker linked to the mutation, and loss of the plasmid marker. The resulting mutant, UNL141, was confirmed with PCR using primers that annealed to the flanking regions.

RT-PCR to Determine hop U1 Expression.

The isolation of DC3000 RNA and reverse transcriptase PCR were performed as in Guo et al. (Guo, M. et al. Pseudomonas syringae type III chaperones ShcO1, ShcS1, and ShcS2 facilitate translocation of their cognate effectors and can substitute for each other in the secretion of HopO1-1. J. Bacteriol. 187:4257-4269 (2005)).

Construction of A. thaliana Col-0 Transgenic Plants Expressing HopU1-HA and HopU1_{DD}-HA.

hopU1 was amplified with PCR and recombined into the Gateway entry vector pENTR/D-TOPO. Site-directed mutagenesis was carried out on this entry construct to change the two glutamic acids in the ADP-RT active site to aspartic acids using the QuikChange Site-Directed Mutagenesis kit (Stratagene). The resulting constructs were recombined into binary Gateway destination vectors such that the hopU1 derivatives were downstream of a CaMV 35S promoter, electroporated into Agrobacterium tumefaciens C58C1, and transformed into A. thaliana Col-0 using the Agrobacterium-mediated floral dip method (Bechtold, N., Ellis, J. & Pelletier, G. In planta Agrobacterium mediated gene transfer by infiltration of adult Arabidopsis thaliana plants. C. R. Acad. Sci. Ser. III Sci. Paris 316:1194-1199 (1993)). Plants were propagated through four generations and confirmed to constitutively produce HopU1-HA or HopU1_{DD}-HA with immunoblots using anti-HA antibodies.

Plant Materials and Bioassays.

P. syringae pv. tomato DC3000 strains were tested for their ability to elicit an HR on Nicotiana tabacum cv. Xanthi by infiltrating strains resuspended in 5 mM MES (pH 5.6) at a cell density of 1×10⁶ or 1×10⁷ cells/ml with a needleless syringe. The ΔhopU1 mutant UNL141 strain carrying plasmids pLN1981 (encoding HopU1), pLN1982 (encoding HOPU1_{DD}), or pML123 (empty vector) were used in HR suppression experiments. Pathogenicity assays with A. thaliana Col-0 and Atgrp7 mutant plants were performed by spray-inoculation with bacterial suspensions that were adjusted to an OD₆₀₀ of 0.2 as described (Espinosa, A., Guo, M., Tam, V. C., Fu, Z. Q. & Alfano, J. R. The Pseudomonas syringae type III-secreted protein HopPtoD2 possesses protein tyrosine phosphatase activity and suppresses programmed cell death in plants. Mol. Microbiol. 49:377-387 (2003)). Bacteria were enumerated from leaf tissue by plating dilutions on KB plates with the appropriate antibiotics. Seeds of Arabidopsis SALK lines SALK_{—}039556.21.25.x and SALK_{—}051743.42.85.x, which were used to identify Atgrp7-1 and Atgrp7-2 mutants, respectively, were obtained from the Ohio State University Biological Resource Center. Further description of methods used to confirm the homozygosity of the T-DNA inserts, and the absence of Atgrp7 RNA and AtGRP7 protein is in the Examples 8 and 9.

Microscopic Detection of Callose Deposition.

A. thaliana Col-0 wild type, Col-0 Atgrp7 mutant plants, and A. thaliana expressing HopU1-HA or HopU1_{DD}-HA were infiltrated with 1 μM of flg22. After 16 h, the leaves were excised, cleared, and stained with aniline blue for callose as described (Gomez-Gomez, L., Felix, G. & Boller, T. A single locus determines sensitivity to bacterial flagellin in Arabidopsis thaliana. Plant J. 18:277-284 (1999)). Leaves were examined with a Zeiss AxioPlan2 Imaging System microscope with fluorescence. The number of callose depositions was determined with Quantity One software (Bio-Rad). Twenty fields of view (each 0.56 mm²) were analyzed and averaged. The average and standard errors of three independent assays for each treatment were recorded.

Purification of HopU1-His and HopU1_{DD}-His mutation derivative.

HopU1-His and HopU1_{DD}-His mutation derivative were affinity-purified from E. coli BL21 (DE3) using Ni-NTA resin following the manufacturer's instructions (Qiagen). Purified HopU1-His and HopU1_{DD}-His were stored in aliquots at −80° C. or on ice. HopU1-His and HopU1_{DD}-His protein concentrations were measured with the BioRad Protein Assay Kit and the purity of the proteins was examined by SDS-PAGE.

Poly-L-Arginine ADP-RT Assay.

To determine if HopU1 ADP-ribosylated the artificial substrate poly-L-arginine we used a previously described protocol (Coye, L. H. & Collins, C. M. Identification of SpyA, a novel ADP-ribosyltransferase of Streptococcus pyogenes. Mol. Microbiol. 54:89-98 (2004)). Briefly, homo-poly-L-arginine (80 μl of 10 mg/ml in 0.1 M dimethyl glutaric acid buffer pH 7.0), 1 μM [³²P]-NAD (GE Healthcare) radiolabeled on the ribose moiety and 1 μM HopU1-His or HopU1_{DD}-His were incubated at room temperature for 1 h, suspended in 0.1 M phosphate buffer, centrifuged, and resuspended in 250 μl of 0.1 M HCl and 500 μl 0.1 M dimethyl glutaric acid buffer (pH 7.0). Incorporated radioactivity was determined using liquid scintillation. BSA (1 μM) was used as a control.

ADP-RT Assays with Plant Extracts and Recombinant Substrates.

The ADP-RT assay was adapted from a described protocol (Sun, J. & Barbieri, J. T. Pseudomonas aeruginosa ExoT ADP-ribosylates CT10 regulator of kinase (Crk) proteins. J. Biol. Chem. 278:32794-32800 (2003)). Briefly, approximately 10 ng of partially-purified HopU1-His was incubated with 5 μCi of [³²P]-NAD (1 Ci/μmole) and A. thaliana Col-0 or tobacco (N. tabacum cv. Xanthi) extracts, E. coli extracts or 1-2 μg of partially purified recombinant substrates. The reaction was stopped by adding SDS sample buffer after incubation at room temperature for 1 h. Samples were subjected to one-dimensional or two-dimensional SDS-PAGE followed by autoradiography of dried gels.

AtGRP7-GST Plasmids and Site-Directed Mutagenesis of AtGRP7-GST.

The nucleotides corresponding to the RRM domain and the glycine-rich domain of AtGRP7 were separately PCR-amplified using primer sets P2578/P2579 and P2580/P2581, respectively, and ligated into the GST vector pGEX-5X- 1. The resulting plasmids encoded an RRM domain-GST fusion (pAtGRP7-RRM) and glycine-rich domain-GST fusion (pAtGRP7-Gly). Several constructs encoding full length AtGRP7-GST fusions in which individual arginine residues in the RRM domain were substituted with lysines were made using the QuikChange Site-Directed Mutagenesis (Stratagene). The primers used to introduce these mutations and the resulting plasmids are listed in Tables 2 and 3. These constructs were separately expressed in E. coli to make lysates that contained AtGRP7-GST derivatives, which were used in ADP-RT assays.

In Planta HopU1 ADP-RT Assays with AtGRP7 using Agrobacterium Delivery.

Agrobacterium transient co-delivery of AtGRP7-HA and hopU1-FLAG or AtGRP7-HA and hopU1_{DD}-FLAG DNA were done as described in Jamir et al (Jamir, Y. et al. Identification of Pseudomonas syringae type III effectors that suppress programmed cell death in plants and yeast. Plant J. 37:554-565 (2004)). After 40 h, plant tissue was harvested with a No. 8 cork borer (12 mm diameter), ground in liquid nitrogen, and resuspended in SDS sample buffer. Samples were subjected to SDS-PAGE and immunoblot analysis using anti-FLAG or anti-HA antibodies to determine the molecular mass of AtGRP7 in the presence or absence of active HopU1-FLAG.

### Example 8

### Bacterial Strains, Media, and Nucleotide Primers

Bacterial strains and plasmids used in this work are listed in Table 2. Escherichia coli strain DH5α was used for general cloning and DNA manipulations and for Gateway technology manipulations E. coli strain DB3.1 was used. Pseudomonas syringae pv. tomato DC3000 was grown in King's B (KB) broth at 30° C. or hrp-inducing fructose minimal medium at 22° C. (King, E. O., Ward, M. K. & Raney, D. E. Two simple media for the demonstration of pyocyanin and fluorescein. J. Lab. Med. 22:301-307 (1954); and Huynh, T. V., Dahlbeck, D. & Staskawicz, B. J. Bacterial blight of soybean: Regulation of a pathogen gene determining host cultivar specificity. Science 245:1374-1377 (1989)). E. coli and Agrobacterium tumefaciens C58C1 were grown in LB broth at 37° C. and 30° C., respectively. Antibiotics were used at the following concentrations (μg ml⁻¹): rifampicin, 100; ampicillin, 100; gentamicin, 10; kanamycin, 50; and spectinomycin 50. Oligonucleotide primers are listed in Table 3.

Adenylate Cyclase Translocation Assays.

The adenylate cyclase assay used was modified from Schechter et al. (Schechter, L. M., Roberts, K. A., Jamir, Y., Alfano, J. R. & Collmer, A. Pseudomonas syringae type III secretion system targeting signals and novel effectors studied with a Cya translocation reporter. J. Bacteriol. 186:543-555 (2004)). Briefly, DC3000 and the DC3000 ΔhrcC type III defective mutant carrying the appropriate constructs, were infiltrated into N. benthamiana leaves at an OD₆₀₀ of 0.6 in 5 mM MES (pH 5.6) containing 100 μM IPTG. Plants were sampled 10 h after infiltration with a 0.8 cm cork-borer. Leaf disks were ground in liquid nitrogen and resuspended in 300 μl 0.1M HCl. Protein concentrations were measured using the Bio-Rad protein assay (Bio-Rad) and cyclic AMP was quantified using the Correlate-EIA Direct cAMP Enzyme Immunoassay kit (Assay Designs) according to the manufacturer's instructions.

Plant Materials and Bioassays.

Pathogenicity assays with A. thaliana Col-0 were performed by spray or dip inoculation with bacterial suspensions that were adjusted to an OD₆₀₀ of 0.2 as described (Espinosa, A., Guo, M., Tam, V. C., Fu, Z. Q. & Alfano, J. R. The Pseudomonas syringae type III-secreted protein HopPtoD2 possesses protein tyrosine phosphatase activity and suppresses programmed cell death in plants. Mol. Microbiol. 49:377-387 (2003)). Bacteria were enumerated from leaf tissue by plating dilutions on KB plates with the appropriate antibiotics. A. thaliana Col-0 plants expressing either HopU1-HA or the catalytic mutant HopU1_{DD}-HA were infiltrated with DC3000 with or without avrRpt2 at 1×10⁷ cells/ml. After 20 h, the plants were photographed and assessed for production of an HR.

Ion Leakage Conductivity Assays.

To determine the amount of ion leakage occurring in plant tissue challenged with DC3000 strains we followed a previously described protocol (Mackey, D., Holt, B. F., III, Wiig, A. & Dangl, J. L. RIN4 interacts with Pseudomonas syringae type III effector molecules and is required for RPM1-mediated resistance in Arabidopsis. Cell 108:743-754 (2002)). Briefly, tobacco (N. tabacum cv. Xanthi) was infiltrated with bacterial strains containing 1×10⁶ or 1×10⁷ cells/ml. After 24 h, three 0.49 cm² leaf discs were removed and floated on 10 ml of fresh water for 1 h. The conductivity of this water was determined with a conductivity meter.

Plasmid Constructions and Site-Directed Mutagenesis of HopU1.

The hopU1 gene was amplified with primers P687 and P688 and recombined into pENTR/D-TOPO Gateway entry vector (Invitrogen) resulting in construct pLN308. The codons corresponding to the catalytic glutamic acid residues of HopU1 in pLN308 were replaced with aspartic acid codons using the QuikChange Site-Directed Mutagenesis Kit (Stratagene) with primers P605 and P606 resulting in construct pLN742. The HopU1-CyaA and HopU1_{DD}-CyaA constructs were made by recombining pLN₃O₈ and pLN742, respectively, into the CyaA Gateway destination vector pCPP3234 using Gateway cloning technology (Invitrogen) to yield pLN919 and pLN920, respectively.

### Example 9

Two-dimensional gel analysis and ion-exchange fractionation of Arabidopsis extracts. Twenty-five g of 6 week old Arabidopsis leaves were collected and ground with sand in 100 ml of 50 mM MOPS, 250 mM sucrose (pH 7.2) with 2 tablets of Complete Protease Inhibitor (Roche). The extract was centrifuged at 12,000 g for 20 m followed by ultracentrifugation at 100,000 g for 30 m. The supernatant was collected and loaded into a Pharmacia FPLC and passed through a 1 ml HiTrap Q XL anion exchange column (GE Healthcare). Forty-five 1 ml fractions were then eluted with a NaCl gradient ranging from 0 to 500 mM in 30 mM MOPS (pH 8.0). ADP-ribosylation assays with HopU1-His were carried out on aliquots of these fractions.

ADP-RT reactions containing either crude Arabidopsis protein extracts or anion-exchange fractions were separated on two-dimensional PAGE gels. The reaction mixtures were precipitated by adding 85% acetone (final concentration, v/v) at −20° C. for at least 2 h. Precipitated proteins were suspended in isoelectric focusing buffer (8 M urea, 2 M thiourea, 2% CHAPS, 2% Triton X-100, and 50 mM DTT). Two hundred μg of Arabidopsis extract were loaded onto 7 cm pH 4-7 ReadyStrip IPG strips (BioRad). After 12 h of active rehydration, proteins were focused using the following program: step 1, 250 V for 15 m; step 2, 8000 V for 1 h (slow voltage ramping); step 3, 8000 V for 30,000 V-h; and step 4, holding at 500 V. The strip was then run in the second dimension using SDS-PAGE and stained with Coomassie blue G-250. Protein spots that had identical migrations as ADP-RT activity spots were cut out and identified using tandem mass spectrometry as described (Rohila, J. S., Chen, M., Cerny, R. & Fromm, M. E. Improved tandem affinity purification tag and methods for isolation of protein heterocomplexes from plants. Plant J. 38:172-181 (2004)).

HopU1-GUS, HopU1-GFP, and Substrate-GFP Fusion Localization in Plant Cells.

Localization of HopU1 and HopU1 substrates were analyzed by transient expression of β-glucuronidase (GUS) or green fluorescent protein (GFP) fusion constructs. GUS-fusions were made in pBI121 (Chen, P. Y., Wang, C. K., Soong, S. C. & To, K. Y. Complete sequence of the binary vector pBI121 and its application in cloning T-DNA insertion from transgenic plants. Mol. Breeding. 11:287-293 (2003)). High quality DNAs were coated on M-17 tungsten particles (Bio-Rad), and bombarded on epidermal layers of onion bulbs using the Biolistic PDS-1000/He System (Bio-Rad). The epidermal cells were allowed to recover on MS plates at room temperature under constant light for 1 day. The epidermal layers were stained for GUS activity as described (Varagona, M. J., Schmidt, R. J. & Raikhel, N. V. Nuclear localization signal(s) required for nuclear targeting of the maize regulatory protein opaque-2. Plant Cell 4:1213-1227 (1992)), and stained cells were observed using a brightfield microscope. HopU1 and HopU1 substrates were fused to GFP in pKFWG2 (Karimi, M., Inze, D. & Depicker, A. GATEWAY vectors for Agrobacterium-mediated plant transformation. Trends Plant Sci. 7:193-195 (2002)) using the Gateway System (Invitrogen). The fusion constructs were transformed into A. tumefaciens C58C1, infiltrated into N. benthamiana leaves, and observed with an Olympus FV500 confocal laser scanning system with an upright microscope.

Characterization of SALK lines SALK_{—}039556.21.25.x and SALK_{—}051743.42.85.x.

Seeds of the SALK T-DNA knock-out line, SALK_{—}039556.21.25.x and SALK_{—}051743.42.85.x were ordered from Arabidopsis Biological Resource Center at Ohio State University. Total genomic DNA was prepared from 4-5 week old individual plants. The homozygosity was confirmed using PCR with genomic DNA from individual plants. For SALK_{—}039556.21.25.x (Atgrp7-1), primer set P2012/P1947 was used to confirm the presence of the T-DNA insertion and primer set P2012/P2013 was used to amplify the wild type AtGRP7 gene. For SALK_{—}051743.42.85x (Atgrp7-2) primer set P2456/P2457 was used to amplify wild type AtGRP7 product and P1947/P2457 was used to confirm the presence of the T-DNA insertion. To confirm that AtGRP7 RNA was not present in the Atgrp7 mutants, total RNA was prepared with Trizol reagent (Invitrogen) following the manufacturer's instructions. cDNAs were amplified from total RNA using RT-PCR at 44° C. with the RETRscript kit (Ambion Inc.) following their instructions. The primer set P1932/P1929 was used to confirm the absence of AtGRP7 mRNA in the homozygous T-DNA Atgrp7-1 and Atgrp7-2 knock-out plants. We used previously described antibodies (Heintzen, C. et al. A light- and temperature-entrained circadian clock controls expression of transcripts encoding nuclear proteins with homology to RNA-binding proteins in meristematic tissue. Plant J. 5:799-813 (1994)) referred to here as anti-GRPs to confirm the Atgrp7 mutants lacked AtGRP7.

### Example 10

### Tables 1-3

TABLE 1
RNA-binding proteins (RBPs) identified with mass spectrometry
(MS) to be ADP-ribosylated by HopU1-His

| | pI and mass | pI and mass | % of protein |
|---|---|---|---|
| ADP-RT Substrate^{a} | (kDa) of | (kD) of ART | coverage by |
| (Accession No.) | protein^{b} | activity spot^{c} | MS peptides^{d} |
| cp29-like Chloroplast | 4.8, 30.7 | 4.5, 25 | 49 |
| RBP (At2g37220.1) | | | |
| cp31-like Chloroplast | 4.6, 31.8 | 4.5, 27 | 37 |
| RBP (At5g50250.1) | | | |
| AtRBP31 Chloroplast | 4.3, 35.7 | 4.3, 32 | 15 |
| RBP (At4g24770.1) | | | |
| AtGRP7 Glycine-rich | 5.9, 16.9 | 5.2, 20 | 27 |
| RBP (At2g21660.1) | | | |
| AtGRP8 Glycine-rich | 5.3, 16.6 | 5.2, 17 | 19 |
| RBP (At4g39260.1) | | | |

^{a}Substrates were isolated based on their identical migration as protein spots that possessed ADP-RT activity and identified with MS.
^{b}Calculated isoelectric points and molecular masses of identified substrates were based on their predicted protein sequence.
^{c}Estimated isoelectric points and molecular masses were based on the migration of protein spots with ADP-RT activity.
^{d}The percent of coverage of the identified protein from the databases with peptides identified from the samples analyzed with MS.

TABLE 2
Bacterial strains and plasmids used in this study

| | | | |
|---|---|---|---|
| | | Primer | |
| | | set used | |
| Designation | Characteristics | in cloning | Reference |

Strains

| | | |
|---|---|---|
| A. tumefaciens | | |
| C58C1 | Disarmed strain for transformation, Rif^{R} Gm^{R} | Van Larebeke, N. et al. |
| | | Large plasmid in |
| | | Agrobacterium tumefaciens |
| | | essential for crown |
| | | gall-inducing ability. |
| | | Nature 252, 169-170 (1974) |
| E. coli | | |
| BL21(DE3) | F⁻ ompT gal [dcm] [lon] hsdS_{B} (r_{B}⁻m_{B}⁻; an E. coli | Novagen |
| | B strain) with DE3, a λ prophage carrying the T7 | |
| | RNA polymerase gene | |
| DB3.1 | F⁻ gyrA462 endA Δ(sr1-recA) mcrB mrr | Invitrogen |
| | hsdS20(r_{B}−m_{B}−) supE44 ara14 galK2 lacY1 proA2 | |
| | rpsL20 (Str^{R}) xyl5λ- leu mtl1 | |
| DH5α | supE44 ΔlacU169(φ80lacZΔM15) hsdR17 recA1 | Life |
| | endA1 gyrA96 thi-1 relA1, Nal^{R} | Technologies |
| P.s. tomato | | |
| DC3000 | Wild type; spontaneous Rif^{R} | Cuppels, D. A. & Ainsworth, |
| | | T. Molecular and physiological |
| | | characterization of |
| | | Pseudomonas syringae pv. |
| | | tomato and |
| | | Pseudomonas syringae pv. |
| | | maculicola strains that |
| | | produce the phytotoxin |
| | | coronatine. Appl. Environ. |
| | | Microbiol. 61, 3530-3536 |
| | | (1995) |
| DC3000-hrcC | DC3000ΔhrcC, secretion deficient mutant, | Yuan, J. & He, S. Y. The |
| | RpCm^{R} | Pseudomonas syringae Hrp |
| | | regulation and secretion |
| | | system controls the production |
| | | and secretion of multiple |
| | | extracellular proteins. |
| | | J. Bacteriol. 178, 6399-6402 |
| | | (1996) |
| UNL141 | DC3000 ΔhopU1 mutant, Rif^{R} Sp^{R} | This work |

Plasmids

| | | | |
|---|---|---|---|
| pAtGRP7-Gly | pGEX-5X-1 derivative that encodes the glycine- | P2580, | This work |
| | rich domain of AtGRP7 fused at its N terminus to | P2581 | |
| | GST, Ap^{R} | | |
| pAtGRP7- | pGEX-5X-1 derivative that encodes the RRM | P2578/ | This work |
| RRM | domain of AtGRP7 fused at its N terminus to | P2579 | |
| | GST, Ap^{R} | | |
| pAvrRpt2-600 | pDSK600 derivative containing avrRpt2, Sp^{R}/Sm^{R} | | Pirhonen, M. U. et al. |
| | | | Phenotypic expression of |
| | | | Pseudomonas syringae avr |
| | | | genes in E. coli is |
| | | | linked to the activities of |
| | | | the hrp-encoded secretion |
| | | | system. Mol Plant-Microbe |
| | | | Interact. 9, 252-260 (1996) |
| pBI121 | Binary vector for Agrobacterium-mediated plant | | Clontech |
| | transformation. Contains 35S promoter and the | | |
| | GUS encoding uidA gene, Km^{R} | | |
| pCPP3234 | Gateway destination vector containing the | | Schechter, L. M., Roberts, |
| | adenylate cyclase (cyaA) gene for C-terminal | | K. A., Jamir, Y., Alfano, |
| | fusions, Sp^{R}/Sm^{R} | | J. R. & Collmer, A. |
| | | | Pseudomonas syringae type |
| | | | III secretion system targeting |
| | | | signals and novel effectors |
| | | | studied with a Cya |
| | | | translocation reporter. |
| | | | J. Bacteriol. 186, 543-555 |
| | | | (2004) |
| pCPP5040 | pML123 derivative converted into a Gateway | | Jamir, Y. et al. |
| | destination vector, Gm^{R} | | Identification of |
| | | | Pseudomonas syringae |
| | | | type III effectors that |
| | | | suppress programmed cell |
| | | | death in plants and yeast. |
| | | | Plant J. 37, 554-565 (2004) |
| pENTR/D- | Gateway system donor vector, Km^{R} | | Invitrogen |
| TOPO | | | |
| pET21(+) | C-terminal His₆-tag expression vector, Ap^{R} | | Novagen |
| pGEX-5X-1 | N-terminal GST-tag expression vector, Ap^{R} | | GE Healthcare |
| pHP45Ω | Vector containing an Sp^{R}/Sm^{R} omega fragment | | Fellay, R., Frey, J. & |
| | with transcriptional and translational terminators, | | Krisch, H. Interposon |
| | Ap^{R} Sp^{R}/Sm^{R} | | mutagenesis of soil and |
| | | | water bacteria: a family of |
| | | | DNA fragments designed for |
| | | | in vitro insertional |
| | | | mutagenesis of Gram-negative |
| | | | bacteria. Gene 52, 147-154 |
| | | | (1987) |
| pK7FWG2 | Gateway binary vector with an N-terminal GFP | | Karimi, M., Inze, D. & |
| | gene for GFP fusions, Sp^{R}/Sm^{R} | | Depicker, A. GATEWAY |
| | | | vectors for Agrobacterium- |
| | | | mediated plant transformation. |
| | | | Trends Plant Sci. 7, |
| | | | 193-195 (2002) |
| pLN308 | pENTR/D-TOPO derivative carrying hopU1, Km^{R} | P687, | This work |
| | | P688 | |
| pLN462 | Gateway destination binary vector for transient | | Jamir, Y. et al. |
| | assays, Sp^{R}/Sm^{R} | | Identification of |
| | | | Pseudomonas syringae |
| | | | type III effectors that |
| | | | suppress programmed cell |
| | | | death in plants and yeast. |
| | | | Plant J. 37, 554-565 (2004) |
| pLN742 | pENTR/D-TOPO derivative carrying hopU1_{DD}, | P605, | This work |
| | Km^{R} | P606 | |
| pLN870 | pET21(+) derivative carrying hopU1, Ap^{R} | P775, | This work |
| | | P1132 | |
| pLN919 | pCPP3234 derivative obtained by recombination | | This work |
| | with pLN308 carrying hopU1, Sp^{R}/Sm^{R} | | |
| pLN920 | pCPP3234 derivative obtained by recombination | | |
| | with pLN742 carrying hopU1_{DD}, Sp^{R}/Sm^{R} | | |
| pLN987 | pET21(+) derivative carrying a hopU1_{DD} | P1132, | This work |
| | mutation, Ap^{R} | P1273 | |
| pLN1072 | pLN462 derivative containing hopU1 made by | | This work |
| | recombination with pLN308, Sp^{R}/Sm^{R} | | |
| pLN1395 | pLN462 derivative containing hopU1_{DD} made by | | This work |
| | recombination with pLN742, Sp^{R}/Sm^{R} | | |
| pLN1492 | pK7FWG2 derivative containing hopU1 made by | | This work |
| | recombination with pLN308, Sp^{R}/Sm^{R} | | |
| pLN1904 | pGEX-5X-1 derivative carrying At2g37220.1 | P1819, | This work |
| | cDNA, Ap^{R} | P1820 | |
| pLN1905 | pGEX-5X-1 derivative carrying At5g50250.1 | P1821, | This work |
| | cDNA, Ap^{R} | P1822 | |
| pLN1981 | pCPP5040 derivative obtained by recombination | | This work |
| | with pLN308, Gm^{R} | | |
| pLN1982 | pCPP5040 derivative obtained by recombination | | This work |
| | with pLN742, Gm^{R} | | |
| pLN1985 | pCPP3234 derivative encoding AvrPto1-CyaA, | | Fu, Z. Q., Guo, M. & Alfano, |
| | Sp^{R}/Sm^{R} | | J. R. Pseudomonas syringae |
| | | | HrpJ is a type III secreted |
| | | | protein that is required for |
| | | | plant pathogenesis, injection |
| | | | of effectors, and secretion |
| | | | of the HrpZ1 harpin. J. |
| | | | Bacteriol. 188, 6060-6069 |
| | | | (2006) |
| pLN1992 | pGEX-5X-1 derivative carrying At4g24770.1 | P1889, | This work |
| | cDNA, Ap^{R} | P1890 | |
| pLN1995 | pENTR/D-TOPO derivative carrying | P1951, | This work |
| | At2g37220.1 cDNA, Km^{R} | P1952 | |
| pLN1998 | pENTR/D-TOPO derivative carrying | P1917, | This work |
| | At4g24770.1 cDNA, Km^{R} | P1918 | |
| pLN1999 | pENTR/D-TOPO derivative carrying | P1923, | This work |
| | At5g50250.1 cDNA, Km^{R} | P1924 | |
| pLN2001 | pK7FWG2 derivative containing At2g37220.1 | | This work |
| | cDNA made by recombination with pLN1995, | | |
| | Sp^{R}/Sm^{R} | | |
| pLN2009 | pGEX-5X-1derivative carrying At4g39260.1 | P1895, | This work |
| | (AtGRP8) cDNA, Ap^{R} | P1896 | |
| pLN2011 | pGEX-5X-1 derivative carrying At2g21660.1 | P1929, | This work |
| | (AtGRP7) cDNA, Ap^{R} | P1932 | |
| pLN2028 | pENTR/D-TOPO derivative carrying hopU1 with | P531, | This work |
| | a C-terminal FLAG fusion, Km^{R} | P687 | |
| pLN2029 | pLN462 derivative containing hopU1 with a C- | | This work |
| | terminal FLAG tag made by recombination with | | |
| | pLN2028, Sp^{R}/Sm^{R} | | |
| pLN2059 | pENTR/D-TOPO derivative carrying | P1925, | This work |
| | At2g21660.1 (AtGRP7) cDNA, Km^{R} | P1926 | |
| pLN2061 | pENTR/D-TOPO derivative carrying | P1927, | This work |
| | At4g39260.1 (AtGRP8) cDNA, Km^{R} | P1928 | |
| pLN2078 | pK7FWG2 derivative containing At2g21660.1 | | This work |
| | (AtGRP7) cDNA made by recombination with | | |
| | PLN2059, Sp^{R}/Sm^{R} | | |
| pLN2079 | pLN462 derivative containing At2g21660.1 | | This work |
| | (AtGRP7) cDNA made by recombination with | | |
| | pLN2059, Sp^{R}/Sm^{R} | | |
| pLN2080 | pK7FWG2 derivative containing At4g39260.1 | | This work |
| | (AtGRPS) cDNA made by recombination with | | |
| | pLN2061, Sp^{R}/Sm^{R} | | |
| pLN2083 | pK7FWG2 derivative containing At5g50250.1 | | This work |
| | cDNA made by recombination with pLN1999, | | |
| | Sp^{R}/Sm^{R} | | |
| pLN2086 | pENTR/D-TOPO derivative carrying hopU1_{DD} | P605, | This work |
| | with a FLAG coding sequence, Km^{R} | P606 | |
| pLN2094 | pBI121 derivative containing hopU1 for HopU1- | | This work |
| | GUS localization, Km^{R} | | |
| pLN2178 | pLN462 derivative containing hopU1_{DD} with a C- | | This work |
| | terminal FLAG fusion made by recombination | | |
| | with pLN2086, Sp^{R}/Sm^{R} | | |
| pLN2256 | pK7FWG2 derivative containing At4g24770.1 | | This work |
| | cDNA made by recombination with pLN1998, | | |
| | Sp^{R}/Sm^{R} | | |
| pLN2433 | pRK415 derivative containing a hopU1 nonpolar | P1058, | This work |
| | deletion mutation, Tc^{R} Sp^{R} | P1059 | |
| | | P1078, | |
| | | P1079 | |
| pLN2593 | pGEX-5X-1 derivative expressing AtGRP7_{R9K}_{^{−}} | P2332, | This work |
| | GST, Ap^{R} | P2333 | |
| pLN2594 | pGEX-5X-1 derivative expressing AtGRP7_{R22K}_{^{−}} | P2334, | This work |
| | GST, Ap^{R} | P2335 | |
| PLN2595 | pGEX-5X-1 derivative expressing AtGRP7_{R43K}_{^{−}} | P2336, | This work |
| | GST, Ap^{R} | P2337 | |
| PLN2596 | pGEX-5X-1 derivative expressing AtGRP7_{R47K}_{^{−}} | P2338, | This work |
| | GST, Ap^{R} | P2339 | |
| pLN2597 | pGEX-5X-1 derivative expressing AtGRP7_{R77K}_{^{−}} | P2340, | This work |
| | GST, Ap^{R} | P2341 | |
| pLN2689 | pGEX-5X-l derivative expressing AtGRP7_{R49K}_{^{−}} | P2488, | This work |
| | GST, Ap^{R} | P2489 | |
| pLN2694 | pGEX-5X-1 derivative expressing AtGRP7_{R87K}_{^{−}} | P2342, | This work |
| | GST, Ap^{R} | P2343 | |
| pML123 | Broad-host-range cloning vector, Gm^{R} Km^{R} | | Labes, M., Puhler, A. & |
| | | | Simon, R. A new family of |
| | | | RSF1010-derived expression |
| | | | and lac-fusion broad-host- |
| | | | range vectors for gram- |
| | | | negative bacteria. Gene 89, |
| | | | 37-46 (1990) |
| pPZP212 | Binary vector for transient expression, Sp^{R}/Sm^{R} | | Hajdukiewicz, P., Svab, Z. & |
| | | | Maliga, P. The small, |
| | | | versatile pPZP family of |
| | | | Agrobacterium binary |
| | | | vectors for plant |
| | | | transformation. Plant Mol. |
| | | | Biol. 25, 989-994 (1994) |
| pPZP212-GFP | Binary vector for transient expression of GFP, | | Qu, F., Ren, T. & Morris, |
| | Sp^{R}/Sm^{R} | | T. J. The coat protein of |
| | | | turnip crinkle virus |
| | | | suppresses posttranscriptional |
| | | | gene silencing at an early |
| | | | initiation step. J. Virol. |
| | | | 77, 511-522 (2003) |
| pRK415 | Broad-host-range vector, unstable in absence of | | Keen, N. T., Tamaski, S., |
| | selection, Tc^{R} | | Kobayashi, D. & Trollinger, |
| | | | D. Improved broad-host-range |
| | | | plasmids for DNA cloning |
| | | | in Gram-negative bacteria. |
| | | | Gene 70, 191-197 (1988) |

TABLE 3
Primer information

| Primer | | Features |
|---|---|---|
| name | Sequences of primer | or uses^{a} |
| P605 | 5′-AAGCGCCCTCAGATGAAGATATCATGCTACCCATG-3′ | HupU1_{DD}-f |
| P606 | 5′-CATGGGTAGCATGATATCTTCATCTGAGGGCGCTT-3′ | HopU1_{DD}-r |
| P687 | 5′-CACCCAACAACCTAATAGTAGAACAATGAATATA-3′ | Gateway |
| P688 | 5′-AATCTGACTTAATACAAATAAATGCTTTCCTT-3′ | Gateway |
| P775 | 5′-CACAAGCTTCGCACATAACAACGGTGA-3′ | HindIII |
| P1058 | 5′-ATGAGGATCCGGATGGGCATGCTCGAAG-3′ | BamHI |
| P1059 | 5′-ATGAAAGCTTAGGCAGTTGTCGATTTAT-3′ | HindIII |
| P1078 | 5′-ATATCTAGAGTCCATGAAGGAGGCCGTACG-3′ | XbaI |
| P1079 | 5′-ATGAGAGCTCGCCTGTCACGACGCCACT-3′ | SacI |
| P1132 | 5′-AGTACTCGAGAATCTGACTTAATACAAA-3′ | XhoI |
| P1247 | 5′-GCAAATAGCGGGTTACTC-3′ | |
| P1248 | 5′-GTCTGGCGCATCGCACAT-3′ | |
| P1273 | 5′-CACAAGCTTCAACAACCTAATAGTAGC-3′ | HindIII |
| P1525 | 5′-CGCGGATCCAAATGAATATAAATCGACAACTG-3′ | BamHI |
| P1528 | 5′-CCGCTCGAGTCAAATCTGACTTAATAC-3′ | XhoI |
| P1819 | 5′-AGTAGAATTCATGGCTGCTTCAGCTTCG-3′ | EcoRI |
| P1820 | 5′-AGTACTCGAGTCAATATTGGCGCCTTGG-3′ | XhoI |
| P1821 | 5′-AGTAGGATCCTGATGACTTCTTCAGTAC-3′ | BamHI |
| P1822 | 5′-AGTACTCGAGTCAACGCCTTGTTCGTTC-3′ | XhoI |
| P1889 | 5′-AGTAGGATCCTGATGGCTTCTTCTATAGTT-3′ | BamHI |
| P1890 | 5′-AGTACTCGAGTTAATATCCACGCCTTGG-3′ | XhoI |
| P1895 | 5′-AGTCGAATTCATGTCTGAAGTTGAGTACCG-3′ | EcoRI |
| P1896 | 5′-AGTCCTCGAGTTACCAGCCGCCACCACCG-3′ | XhoI |
| P1917 | 5′-CACCAACAATGGCTTCTTCTATAGTT-3′ | Gateway |
| P1918 | 5′-ATATCCACGCCTTGGAGG-3′ | Gateway |
| P1923 | 5′-CACCAACAATGACTTCTTCAGTACTA-3′ | Gateway |
| P1924 | 5′-ACGCCTTGTTCGTTCCTC-3′ | Gateway |
| P1925 | 5′-CACCAACAATGGCGTCCGGTGATGTT-3′ | Gateway |
| P1926 | 5′-CCATCCTCCACCACCACC-3′ | Gateway |
| P1927 | 5′-CACCAACAATGTCTGAAGTTGAGTAC-3′ | Gateway |
| P1928 | 5′-CCAGCCGCCACCACCGCC-3′ | Gateway |
| P1929 | 5′-AGTCCTCGAGTTACCATCCTCCACCACC-3′ | XhoI |
| P1932 | 5′-CACCGAATTCATGGCGTCCGGTGATGTTG-3′ | EcoRI |
| P1947 | 5′-TGGTTCACGTAGTGGGCCATCG-3′ | |
| P1951 | 5′-CACCATGGCTGCTTCAGCTTCGTC-3′ | Gateway |
| P1952 | 5′-ATATTGGCGCCTTGGAGGCC-3′ | Gateway |
| P1956 | 5′-AGATTCTAGAATGAATATAAATCGACAACTGC-3′ | XbaI |
| P1957 | 5′-AGATGGATCCAATCTGACTTAATACAAATAAATGC-3′ | BamHI |
| P2012 | 5′-GCCAGATTAAGGTTCTGG-3′ | |
| P2013 | 5′-TCGATATCACTCGGAGTCCG-3′ | |
| P2332 | 5′-CGTCCGGTGATGTTGAGTATAAATGCTTCGTTGGAGGTCTAGC-3′ | AtGRP7_{R9K}-f |
| P2333 | 5′-GCTAGACCTCCAACGAAGCATTTATACTCAACATCACCGGACG-3′ | AtGRP7_{R9K}-r |
| P2334 | 5′-GCATGGGCCACTGATGACAAAGCTCTTGAGACTGCCTTCG-3′ | AtGRP7_{R22K}-f |
| P2335 | 5′-CGAAGGCAGTCTCAAGAGCTTTGTCATCAGTGGCCCATG-3′ | AtGRP7_{R22K}-r |
| P2336 | 5′-CCAAGATCATTAACGATAAAGAGACTGGAAGATCAAGG-3′ | AtGRP7_{R43K}-f |
| P2337 | 5′-CCTTGATCTTCCAGTCTCTTTATCGTTAATGATCTTGG-3′ | AtGRP7_{R43K}-r |
| P2338 | 5′-CGATCGTGAGACTGGAAAATCAAGGGGATTCGGATTCG-3′ | AtGRP7_{R47K}-f |
| P2339 | 5′-CGAATCCGAATCCCCTTGATTTTCCAGTCTCACGATCG-3′ | AtGRP7_{R47K}-r |
| P2340 | 5′-GGACAAGATCTCGATGGCAAAAGCATCACTGTTAACGAGG-3′ | AtGRP7_{R77K}-f |
| P2341 | 5′-CCTCGTTAACAGTGATGCTTTTGCCATCGAGATCTTGTCC-3′ | AtGRP7_{R77K}-r |
| P2342 | 5′-GTTAACGAGGCTCAGTCAAAAGGAAGCGGTGGCGGCGG-3′ | AtGRP7_{R87K}-f |
| P2343 | 5′-CCGCCGCCACCGCTTCCTTTTGACTGAGCCTCGTTAAC-3′ | AtGRP7_{R87K}-r |
| P2456 | 5′-ATTGATCCGGCCCAGTATCAGTTGT-3′ | |
| P2457 | 5′-CCAGATCAGCAAAACAATCC-3′ | |
| p2488 | 5′-CGTGAGACTGGAAGATCAAAAGGATTCGGATTCGTCACC-3′ | AtGRP7_{R49K}-f |
| p2489 | 5′-GGTGACGAATCCGAATCCTTTTGATCTTCCAGTCTCACG-3′ | AtGRP7_{R49K}-r |
| p2578 | 5′-CGCGAATTCATGGCGTCCGGTGATG-3′ | AtGRP7 RRM-f |
| p2579 | 5′-AAGAATGCGGCCGCTAATCGTGACTGAGCCTCG-3′ | AtGRP7 RRM-r |
| p2580 | 5′-GCGAATTCCAGTCACGAGGAAGC-3′ | AtGRP7 Gly-f |
| p2581 | 5′-GGGATCCTTACCATCCTCCACC-3′ | AtGRP7 Gly-r |

^{a}Indicates whether the primer contained a restriction enzyme site, was used in Gateway recombination cloning, or site-directed mutatgenesis of HopU1 or AtGRP7.

What is claimed is:

1. A method of enhancing an innate immune response of a plant comprising:
over-expressing in the plant a HopU1 substrate capable of being ADP-ribosylated by HopU1.

2. The method of claim 1, further comprising introducing into a plant cell a construct comprising a polynucleotide encoding the HopU1 substrate, wherein the HopU1 substrate polynucleotide is operably linked to a promoter functional in plant cells to yield transformed plant cells; and
regenerating a transgenic plant from the transformed plant cells, wherein the HopU1 substrate is expressed in the cells of the transgenic plant at levels sufficient to enhance the immune response in the transgenic plant.

3. The method of claim 1, wherein the HopU1 substrates are RNA binding proteins.

4. The method of claim 3, wherein the HopU1 substrates comprise Glycine rich RNA binding proteins or Chloroplast RNA binding proteins.

5. The method of claim 1, wherein the HopU1 substrates are mutated in their ADP-ribosylation sites.

6. The method of claim 1, the biotic stresses comprise a pathogen, bacteria, fungi, protozoa a virus, an insect, wounding, or physical damage caused by people.

7. The method of claim 1, wherein the plant undergoes a hypersensitive response and cells in contact with the biotic stress die.

8. The method of claim 1, further comprising identifying a substrate of HopU1 and introducing the substrate into the plant cell.

9. The method of claim 8, further comprising identifying one or more HopU1 substrates by incubating a candidate substrate of HopU1 in the presence of HopU1 and assaying the substrate to determine if the substrate underwent ADP-ribosylation.

10. The method of claim 1, wherein the plant is a dicotyledonous plant.

11. The method of claim 1, wherein the plant is a monocotyledonous plant.

12. The method of claim 1, wherein the plant is corn, tomato, soybean, sunflower, sorghum, canola, wheat, alfalfa, cotton, rice, barley or millet.

13. The method of claim 1, wherein immune response is enhanced as compared to a control plant, wherein the HopU1 substrate is not over-expressed in the control plant.

14. A method of identifying a substrate of HopU1 in a plant or animal cell comprising:
incubating an extract from a plant or animal cell suspected of comprising a candidate HopU1 substrate in the presence of HopU1 and assaying the substrate to determine if the candidate HopU1 substrate underwent ADP-ribosylation.

15. The method of claim 14, wherein the candidate HopU1 substrate is a RNA binding protein.

16. The method of claim 15, wherein the RNA binding protein is a glycine rich RNA binding protein.

17. The method of claim 14, wherein HopU1 ribosylates the candidate HopU1 substrate in the presence of NAD⁺.

18. The method of claim 14, wherein the assaying further comprises separating the extract on a SDS-PAGE gel and using autoradiography to determine a molecular mass of the candidate HopU1 substrate.


SEQUENCE LISTING (93 sequences)

SEQ ID NO: 1 - DNA, 35 bp, Artificial
  Artificial sequence - Primer
aagcgccctcagatgaagatatcatgctacccatg

SEQ ID NO: 2 - DNA, 35 bp, Artificial sequence
  Artifical sequence - Primer
catgggtagcatgatatcttcatctgagggcgctt

SEQ ID NO: 3 - DNA, 34 bp, Artificial sequence
  Artifical sequence - Primer
cacccaacaacctaatagtagaacaatgaatata

SEQ ID NO: 4 - DNA, 33 bp, Artificial sequence
  Artifical sequence - Primer
aatctgacttaatacaaataaatgctttccttc

SEQ ID NO: 5 - DNA, 27 bp, Artificial sequence
  Artifical sequence - Primer
cacaagcttcgcacataacaacggtga

SEQ ID NO: 6 - DNA, 28 bp, Artificial sequence
  Artifical sequence - Primer
atgaggatccggatgggcatgctcgaag

SEQ ID NO: 7 - DNA, 28 bp, Artificial sequence
  Artifical sequence - Primer
atgaaagcttaggcagttgtcgatttat

SEQ ID NO: 8 - DNA, 30 bp, Artificial sequence
  Artifical sequence - Primer
atatctagagtccatgaaggaggccgtacg

SEQ ID NO: 9 - DNA, 28 bp, Artificial sequence
  Artifical sequence - Primer
atgagagctcgcctgtcacgacgccact

SEQ ID NO: 10 - DNA, 28 bp, Artificial sequence
  Artifical sequence - Primer
agtactcgagaatctgacttaatacaaa

SEQ ID NO: 11 - DNA, 18 bp, Artificial sequence
  Artifical sequence - Primer
gcaaatagcgggttactc

SEQ ID NO: 12 - DNA, 18 bp, Artificial sequence
  Artifical sequence - Primer
gtctggcgcatcgcacat

SEQ ID NO: 13 - DNA, 26 bp, Artificial sequence
  Artifical sequence - Primer
cacaagcttcaacaacctaatagtag

SEQ ID NO: 14 - DNA, 33 bp, Artificial sequence
  Artifical sequence - Primer
cgcggatccaaatgaatataaatcgacaactgc

SEQ ID NO: 15 - DNA, 27 bp, Artificial sequence
  Artifical sequence - Primer
ccgctcgagtcaaatctgacttaatac

SEQ ID NO: 16 - DNA, 28 bp, Artificial sequence
  Artifical sequence - Primer
agtagaattcatggctgcttcagcttcg

SEQ ID NO: 17 - DNA, 28 bp, Artificial sequence
  Artifical sequence - Primer
agtactcgagtcaatattggcgccttgg

SEQ ID NO: 18 - DNA, 28 bp, Artificial sequence
  Artifical sequence - Primer
agtaggatcctgatgacttcttcagtac

SEQ ID NO: 19 - DNA, 28 bp, Artificial sequence
  Artifical sequence - Primer
agtactcgagtcaacgccttgttcgttc

SEQ ID NO: 20 - DNA, 30 bp, Artificial sequence
  Artifical sequence - Primer
agtaggatcctgatggcttcttctatagtt

SEQ ID NO: 21 - DNA, 28 bp, Artificial sequence
  Artifical sequence - Primer
agtactcgagttaatatccacgccttgg

SEQ ID NO: 22 - DNA, 30 bp, Artificial sequence
  Artifical sequence - Primer
agtcgaattcatgtctgaagttgagtaccg

SEQ ID NO: 23 - DNA, 29 bp, Artificial sequence
  Artifical sequence - Primer
agtcctcgagttaccagccgccaccaccg

SEQ ID NO: 24 - DNA, 26 bp, Artificial sequence
  Artifical sequence - Primer
caccaacaatggcttcttctatagtt

SEQ ID NO: 25 - DNA, 18 bp, Artificial sequence
  Artifical sequence - Primer
atatccacgccttggagg

SEQ ID NO: 26 - DNA, 26 bp, Artificial sequence
  Artifical sequence - Primer
caccaacaatgacttcttcagtacta

SEQ ID NO: 27 - DNA, 18 bp, Artificial sequence
  Artifical sequence - Primer
acgccttgttcgttcctc

SEQ ID NO: 28 - DNA, 26 bp, Artificial sequence
  Artifical sequence - Primer
caccaacaatggcgtccggtgatgtt

SEQ ID NO: 29 - DNA, 18 bp, Artificial sequence
  Artifical sequence - Primer
ccatcctccaccaccacc

SEQ ID NO: 30 - DNA, 26 bp, Artificial sequence
  Artifical sequence - Primer
caccaacaatgtctgaagttgagtac

SEQ ID NO: 31 - DNA, 18 bp, Artificial sequence
  Artifical sequence - Primer
ccagccgccaccaccgcc

SEQ ID NO: 32 - DNA, 28 bp, Artificial sequence
  Artifical sequence - Primer
agtcctcgagttaccatcctccaccacc

SEQ ID NO: 33 - DNA, 29 bp, Artificial sequence
  Artifical sequence - Primer
caccgaattcatggcgtccggtgatgttg

SEQ ID NO: 34 - DNA, 22 bp, Artificial sequence
  Artifical sequence - Primer
tggttcacgtagtgggccatcg

SEQ ID NO: 35 - DNA, 24 bp, Artificial sequence
  Artifical sequence - Primer
caccatggctgcttcagcttcgtc

SEQ ID NO: 36 - DNA, 20 bp, Artificial sequence
  Artifical sequence - Primer
atattggcgccttggaggcc

SEQ ID NO: 37 - DNA, 32 bp, Artificial sequence
  Artifical sequence - Primer
agattctagaatgaatataaatcgacaactgc

SEQ ID NO: 38 - DNA, 35 bp, Artificial sequence
  Artifical sequence - Primer
agatggatccaatctgacttaatacaaataaatgc

SEQ ID NO: 39 - DNA, 18 bp, Artificial sequence
  Artifical sequence - Primer
gccagattaaggttctgg

SEQ ID NO: 40 - DNA, 20 bp, Artificial sequence
  Artifical sequence - Primer
tcgatatcactcggagtccg

SEQ ID NO: 41 - DNA, 43 bp, Artificial sequence
  Artifical sequence - Primer
cgtccggtgatgttgagtataaatgcttcgttggaggtctagc

SEQ ID NO: 42 - DNA, 43 bp, Artificial sequence
  Artifical sequence - Primer
gctagacctccaacgaagcatttatactcaacatcaccggacg

SEQ ID NO: 43 - DNA, 40 bp, Artificial sequence
  Artifical sequence - Primer
gcatgggccactgatgacaaagctcttgagactgccttcg

SEQ ID NO: 44 - DNA, 40 bp, Artificial sequence
  Artifical sequence - Primer
cgaaggcagtctcaagagctttgtcatcagtggcccatgc

SEQ ID NO: 45 - DNA, 38 bp, Artificial sequence
  Artifical sequence - Primer
ccaagatcattaacgataaagagactggaagatcaagg

SEQ ID NO: 46 - DNA, 38 bp, Artificial sequence
  Artifical sequence - Primer
ccttgatcttccagtctctttatcgttaatgatcttgg

SEQ ID NO: 47 - DNA, 38 bp, Artificial sequence
  Artifical sequence - Primer
cgatcgtgagactggaaaatcaaggggattcggattcg

SEQ ID NO: 48 - DNA, 38 bp, Artificial sequence
  Artifical sequence - Primer
cgaatccgaatccccttgattttccagtctcacgatcg

SEQ ID NO: 49 - DNA, 40 bp, Artificial sequence
  Artifical sequence - Primer
ggacaagatctcgatggcaaaagcatcactgttaacgagg

SEQ ID NO: 50 - DNA, 40 bp, Artificial sequence
  Artifical sequence - Primer
cctcgttaacagtgatgcttttgccatcgagatcttgtcc

SEQ ID NO: 51 - DNA, 38 bp, Artificial sequence
  Artifical sequence - Primer
gttaacgaggctcagtcaaaaggaagcggtggcggcgg

SEQ ID NO: 52 - DNA, 38 bp, Artificial sequence
  Artifical sequence - Primer
ccgccgccaccgcttccttttgactgagcctcgttaac

SEQ ID NO: 53 - DNA, 25 bp, Artificial sequence
  Artifical sequence - Primer
attgatccggcccagtatcagttgt

SEQ ID NO: 54 - DNA, 20 bp, Artificial sequence
  Artifical sequence - Primer
ccagatcagcaaaacaatcc

SEQ ID NO: 55 - DNA, 39 bp, Artificial sequence
  Artifical sequence - Primer
cgtgagactggaagatcaaaaggattcggattcgtcacc

SEQ ID NO: 56 - DNA, 39 bp, Artificial sequence
  Artifical sequence - Primer
ggtgacgaatccgaatccttttgatcttccagtctcacg

SEQ ID NO: 57 - DNA, 25 bp, Artificial sequence
  Artifical sequence - Primer
cgcgaattcatggcgtccggtgatg

SEQ ID NO: 58 - DNA, 34 bp, Artificial sequence
  Artifical sequence - Primer
taagaatgcggccgctaatcgtgactgagcctcg

SEQ ID NO: 59 - DNA, 23 bp, Artificial sequence
  Artifical sequence - Primer
gcgaattccagtcacgaggaagc

SEQ ID NO: 60 - DNA, 22 bp, Artificial sequence
  Artifical sequence - Primer
gggatccttaccatcctccacc

SEQ ID NO: 61 - protein, 13 aa, Pseudomonas syringae
GTPLYREVNNYLR

SEQ ID NO: 62 - protein, 19 aa, Pseudomonas syringae
GGRYVEPAFMSTTRIKDSA

SEQ ID NO: 63 - protein, 14 aa, Pseudomonas syringae
ISGSSQAPSEEEIM

SEQ ID NO: 64 - protein, 13 aa, Pseudomonas syringae
TSCLYRPINHHLR

SEQ ID NO: 65 - protein, 19 aa, Pseudomonas syringae
GNTYRDDAFMSTSTRMDVT

SEQ ID NO: 66 - protein, 14 aa, Pseudomonas syringae
IGPFSKNPYEDEAL

SEQ ID NO: 67 - protein, 13 aa, Pseudomonas syringae
TSCLYRPINHHLR

SEQ ID NO: 68 - protein, 19 aa, Pseudomonas syringae
GKTYRDEAFMSTSTHMQVS

SEQ ID NO: 69 - protein, 14 aa, Pseudomonas syringae
IGPFSKNPYEDEAL

SEQ ID NO: 70 - protein, 13 aa, Pseudomonas syringae
GDRLYRADSRPPD

SEQ ID NO: 71 - protein, 19 aa, Pseudomonas syringae
GFVRYDDGYVSTSLSLRSA

SEQ ID NO: 72 - protein, 14 aa, Pseudomonas syringae
LGVYSPHPYEQEVS

SEQ ID NO: 73 - protein, 13 aa, Pseudomonas syringae
DDKLYRADSRPPD

SEQ ID NO: 74 - protein, 19 aa, Pseudomonas syringae
GFVRHDDGYVSTSISLRSA

SEQ ID NO: 75 - protein, 14 aa, Pseudomonas syringae
LGAYSPHPDEQEVS

SEQ ID NO: 76 - protein, 13 aa, Pseudomonas syringae
CYYVYRGVRGIRF

SEQ ID NO: 77 - protein, 19 aa, Pseudomonas syringae
GKSVYFGQFTSTSLRKEAT

SEQ ID NO: 78 - protein, 14 aa, Pseudomonas syringae
IKQFSFFPSEDEVL

SEQ ID NO: 79 - protein, 13 aa, Pseudomonas syringae
DMKVYRGTDLNPL

SEQ ID NO: 80 - protein, 19 aa, Pseudomonas syringae
GKTFKDDGFMSTALVKESS

SEQ ID NO: 81 - protein, 14 aa, Pseudomonas syringae
VSKISYFPDEAELL

SEQ ID NO: 82 - protein, 13 aa, Pseudomonas syringae
HRVVYRGLKLDPK

SEQ ID NO: 83 - protein, 19 aa, Pseudomonas syringae
GNIIIDKAFMSTSPDKAWI

SEQ ID NO: 84 - protein, 14 aa, Pseudomonas syringae
LGDVAHFKGEAEML

SEQ ID NO: 85 - protein, 13 aa, Pseudomonas syringae
NITVYRWCGMPEF

SEQ ID NO: 86 - protein, 19 aa, Pseudomonas syringae
NTIKEDKGYMSTSLSSERL

SEQ ID NO: 87 - protein, 14 aa, Pseudomonas syringae
LSAIGGFASEKEIL

SEQ ID NO: 88 - protein, 13 aa, Pseudomonas syringae
VVKTFRGTRGGDA

SEQ ID NO: 89 - protein, 19 aa, Pseudomonas syringae
GKVGHDDGYLSTSLNPGVA

SEQ ID NO: 90 - protein, 14 aa, Pseudomonas syringae
VSGISNYKNEKEIL

SEQ ID NO: 91 - protein, 13 aa, Pseudomonas syringae
VVKTFRGTQGRDA

SEQ ID NO: 92 - protein, 19 aa, Pseudomonas syringae
GQVGHDAGYLSTSRDPGVA

SEQ ID NO: 93 - protein, 14 aa, Pseudomonas syringae
VSEISIEGDEQEIL